(19) United States
(12) Patent Application Publication
(10) Pub. No.: US 2008/0081078 A1
(43) Pub. Date: Apr. 3, 2008

(54) Prevention of Cell Proliferation by Inhibiting Myosin Light Chain Kinase

(75) Inventor: Primal de Lanerolle, Oak Park, IL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: The Board of Trustees of the University of Illinois, Urbana, IL (US)

(21) Appl. No.: 11/694,484

(22) Filed: Mar. 30, 2007

Related U.S. Application Data
(60) Provisional application No. 60/822,349, filed on Aug. 14, 2006.

(51) Int. Cl. A61K 31/551; A61K 31/00; A61K 31/04; A61K 31/05; A61K 31/265; A61K 31/282; A61K 31/337; A61K 31/70; A61K 38/00; C12Q 1/02; A61P 35/00; A61K 38/16; A61K 33/24; A61K 31/566; A61K 31/4164; A61K 31/44; A61K 31/47; A61K 31/497; A61K 31/505

(52) U.S. Cl. 424/649; 435/29; 514/177; 514/2; 514/211.08; 514/249; 514/253.04; 514/274; 514/283; 514/310; 514/34; 514/398; 514/449; 514/512; 514/648; 514/672; 514/733; 514/789; 514/8

(57) ABSTRACT

Methods of using compounds having certain inhibitory effect to treat cancer are disclosed. More specifically, methods of using myosin light chain kinase inhibitor (MLCK) to treat cancer are disclosed. MLCK inhibitors may cause reduction in MLC-P and induce apoptosis in neoplastic cells and prevent and or inhibit the tumor growth.

### STATEMENT OF RELATED CASES

This application claims priority to U.S. Provisional Application Ser. No. 60/822,349, filed Aug. 14, 2006, the entirety of which is incorporated by reference herein.

### FEDERALLY SPONSORED RESEARCH OR DEVELOPMENT

The subject matter of this application was funded by the National Institute of Health (Grant nos. NIH HL 59618 and NIH HL 0241 1). The government may have certain rights in this invention. Any opinions, findings, and conclusions or recommendations expressed in this publication do not necessarily reflect the views of the National Institutes of Health.

### BACKGROUND OF THE INVENTION

1. Technical Field of the Invention

The present invention relates to the methods of treating cancer and other proliferative diseases. More specifically, the present invention relates to administering compounds having certain inhibitory effects to prevent or inhibit tumor growth.

2. Background of the Related Art

Cancer is one of the leading causes of death in the United States and over 8,000,000 persons in the United States have been diagnosed with cancer. In 1995, cancer accounted for 23.3% of all deaths in the United States. (See U.S. Dept. of Health and Human Services, National Center for Health Statistics, Health United States 1996-97 and Injury Chartbook 11 7 (1997)). Cancer is not fully understood on the molecular level. It is known that exposure of a cell to a carcinogen such as certain viruses, certain chemicals, or radiation, leads to DNA alteration that inactivates a “suppressive” gene or activates an “oncogene”. Suppressive genes are growth regulatory genes, which upon mutation, can no longer control cell growth. Oncogenes are initially normal genes (called protooncogenes) that by mutation or altered context of expression become transforming genes. The products of transforming genes cause inappropriate cell growth. More than twenty different normal cellular genes can become oncogenes by genetic alteration. Transformed cells differ from normal cells in many ways, including cell morphology, cell-to-cell interactions, membrane content, cytoskeletal structure, protein secretion, gene expression and mortality (transformed cells can grow indefinitely).

A neoplasm, or tumor, is an abnormal, unregulated, and disorganized proliferation of cell growth, and is generally referred to as cancer. A neoplasm is malignant, or cancerous, if it has properties of destructive growth, invasiveness and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Cancer is now primarily treated with one or a combination of three types of therapies: surgery, radiation, and chemotherapy.

Surgery involves the bulk removal of diseased tissue. While surgery is sometimes effective in removing tumors located at certain sites, for example, in the breast, colon, and skin, it cannot be used in the treatment of tumors located in other areas, such as the backbone, nor in the treatment of disseminated neoplastic conditions such as leukemia.

Radiation therapy involves the exposure of living tissue to ionizing radiation causing death or damage to the exposed cells. Side effects from radiation therapy may be acute and temporary, while others may be irreversible.

Chemotherapy involves the disruption of cell replication or cell metabolism. It is used most often in the treatment of breast, lung, and testicular cancer.

The adverse effects of systemic chemotherapy used in the treatment of neoplastic disease are most feared by patients undergoing treatment for cancer. Of these adverse effects nausea and vomiting are the most common and severe side effects. Other adverse side effects include cytopenia, infection, cachexia, mucositis in patients receiving high doses of chemotherapy with bone marrow rescue or radiation therapy; alopecia (hair loss); cutaneous complications (see M. D. Abeloff et al., Alopecia and Cutaneous Complications, p. 755-56 in Abeloff, M. D., Armitage, J. O., Lichter, A. S., and Niederhuber, J. E. (eds), Clinical Oncology, Churchill Livingston, N.Y., 1992, for cutaneous reactions to chemotherapy agents), such as pruritis, urticaria, and angioedema; neurological complications; pulmonary and cardiac complications in patients receiving radiation or chemotherapy; and reproductive and endocrine complications. Chemotherapy-induced side effects significantly impact the quality of life of the patient and may dramatically influence patient compliance with treatment.

Additionally, adverse side effects associated with chemotherapeutic agents are generally the major dose-limiting toxicity (DLT) in the administration of these drugs. For example, mucositis is one of the major dose-limiting toxicity for several anticancer agents, including the antimetabolite cytotoxic agents 5-FU, methotrexate, and antitumor antibiotics, such as doxorubicin. Many of these chemotherapy-induced side effects if severe, may lead to hospitalization, or require treatment with analgesics for the treatment of pain.

Adverse side effects induced by anticancer therapy have become of major importance to the clinical management of cancer patients undergoing treatment for cancer or neoplasia disease.

As set forth above, untreated cancer may result in severe consequences, including death. Clearly, new treatment methods and agents are needed and would be welcomed by those plagued by cancer who either cannot tolerate available treatment regimens or undergo invasive surgical procedures.

Accordingly, effective methods of treatment and compositions capable of preventing or eliminating tumors would greatly aid in current cancer treatment methods.

### SUMMARY OF THE INVENTION

In one embodiment the invention is a method of inhibiting a tumor growth in a subject. The method comprises administering to the subject a pharmaceutical composition comprising a compound in an amount sufficient to cause a reduction in myosin light chain phosphorylation (MLC-P) during apoptosis of the cells of the tumor, and thereby inhibiting the tumor growth.

In one embodiment the invention is a method of treating a patient having neoplasia. The method comprises administering to the patient in need thereof a pharmaceutical composition comprising a therapeutically effective amount of at least one myosin light chain kinase inhibitor in a therapeutically effective amount sufficient to cause a reduction in MLC-P during apoptosis of the neoplastic cells in the patient.

In another embodiment the invention is a method of inducing apoptosis in neoplastic cells in a subject. The method comprises administering to the subject a composition comprising at least one myosin light chain kinase inhibitor in a dose effective to induce apoptosis in the neoplastic cells.

In a further embodiment, the present invention is a method of inducing apoptosis in tumor cells in a subject. The method comprises administering to the subject a composition comprising an effective amount of at least one myosin light chain kinase inhibitor and thereby inducing apoptosis in the tumor cells. The method further comprises evaluating the cells for indication of apoptosis.

In another embodiment, the invention is a method of inhibiting a tumor growth in a subject. The method comprises administering to the subject at least one myosin light chain kinase inhibitor in the amount sufficient to cause a reduction in MLC-P during apoptosis of the cells of the tumor, and thereby inhibiting the tumor growth.

In yet another embodiment, the present invention is a method of treating a patient having neoplasia. The method includes administering to the patient in need thereof a pharmaceutical composition comprising at least one cytotoxic drug. The method further comprises administering to the patient in need thereof a pharmaceutical composition comprising at least one myosin light chain kinase inhibitor. The compositions are administered in the amounts which, together, are effective to cause a reduction in MLC-P during apoptosis on the neoplastic cells in the patient.

In a further embodiment, the present invention is a method of inhibiting growth or proliferation of, or inducing reduction in the number of tumor cells in a subject, comprising administering to the subject at least one cytotoxic agent and at least one myosin light chain kinase (MLCK) inhibitor, in an amount which, together, is effective to cause a reduction in MLC-P and inhibit growth or proliferation of the tumor cells.

Also, in another embodiment, the present invention is a method of inhibiting unwanted growth or proliferation of, or reducing the number of tumor cells in a human subject. The method includes administering to the human subject at least one cytotoxic agent and at least one MLCK inhibitor, in an amount, which together, is effective to cause a reduction in MLC-P and reduce or inhibit the growth or proliferation of the established tumor, induce cell death of the established tumors, or to reduce the size of the established tumors.

In yet another embodiment, the present invention is a method of inhibiting unwanted proliferation of, or reducing the size of, an established tumor in a subject. The method comprises administering to the subject at least one cytotoxic agent and at least one MLCK inhibitor, in an amount, which together, is effective to cause a reduction in MLC-P and reduce or inhibit the growth or proliferation of the established tumor, induce cell death of the established tumors, or to reduce the size of the established tumors. The established tumor is breast cancer, prostate cancer, lung carcinoma, renal cell carcinoma, glioma, melanoma, chemotherapy resistant tumors or metastatic tumors.

In another embodiment, the present invention is a method for identifying a compound with potential for treating neoplasia. The method includes determining an MLC-P level in a first mammalian cell after exposing the cell to the compound and determining whether the compound induces apoptosis of a second mammalian cell. The method further includes a step of identifying the compound as having potential for treating neoplasia when reduced MLC-P levels and induced apoptosis are determined.

In yet another embodiment, the present invention is a method of treating a patient having neoplasia. The method includes administering to the patient in need thereof a pharmaceutical composition comprising at least one compound, identified as a compound with potential for treating neoplasia. The compound is administered in a therapeutically effective amount sufficient to cause a reduction in MLC-P during apoptosis of the neoplastic cells in the patient.

Specific preferred embodiments of the present invention will become evident from the following more detailed description of certain preferred embodiments and the claims.

### BRIEF DESCRIPTION OF THE DRAWINGS

FIGS. 1A and 1B show Western blots, a line graph (FIG. 1A) and a bar graph (FIG. 1B) depicting phosphorylation of the 20 kD light chain of myosin II (MLC₂₀) and caspase activity during apoptosis.

FIGS. 2A and 2B show a Western blot and bar graphs depicting MLC₂₀ dephosphorylation and apoptosis due to inhibition of MLCK.

FIGS. 3A and 3B show a line graph (FIG. 3A) and photomicrographs (FIG. 3B) depicting MLC₂₀ dephosphorylation and apoptosis due to inhibition of MLCK.

FIGS. 4A and 4B show a Western blot and a bar graph depicting a distribution of MLC forms (FIG. 4A) and an increase of cell death as a result of cytochalasin D (CytoD) treatment as compared to untreated controls (FIG. 4B).

FIGS. 5A and 5B show a bar graph (FIG. 5A) and photomicrographs (FIG. 5B) depicting protection against apoptosis due to capase inhibition. There was a decrease in apoptosis induced by MLCK inhibition in cells that were treated with a caspase inhibitor (z-VAD-fmk) as compared to cells that were not treated with a caspase inhibitor.

FIGS. 6A and 6B show bar graphs depicting the effect of ML-7 on apoptosis in Mm5MT and MLL cells.

FIG. 7 is a line graph depicting ML-7 potentiating the induction of apoptosis in MLL prostate cancer cells by etoposide.

FIG. 8 is a bar graph depicting ML-7 potentiating the induction of apoptosis in Mm5MT breast cancer cells by etoposide.

FIG. 9 is a photograph of Western blot analysis (inset) and a bar graph depicting ML-7 potentiating the induction of apoptosis in Mm5MT cells by etoposide.

FIG. 10 is a bar graph depicting the chemopreventive effect of ML-7 in mouse mammary gland organ culture.

FIG. 11 shows photomicrographs demonstrating the presence of more apoptotic cells in the tissue sections from the mice receiving ML-7 than in the controls.

FIG. 12 shows photographs depicting a decrease in tumor size following the treatment with myosin light chain kinase inhibitor ML-7 (bottom) as compared to the tumor removed from an untreated mouse (top).

FIG. 13A is a photograph depicting a decrease in tumor size following the treatment with ML-7, etoposide, and ML-7+etoposide as compared to control (a tumor removed from an untreated mouse).

FIG. 13B is a bar graph depicting tumor weight from animals treated with ML-7, etoposide, and ML-7+etoposide as compared to control.

FIG. 14A is a bar graph depicting percent of viable tumor area from animals treated with ML-7, etoposide, and ML-7+etoposide compared with control.

FIG. 14B shows photomicrographs depicting limited areas of necrosis present in tumors from etoposide and ML-7 treated mice as compared to control mice.

FIG. 15 shows a photograph image of Western blot analysis (inset) and a bar graph depicting the ability of ML-7 to stimulate the ability of etoposide to induce apoptosis.

FIG. 16A is a photograph depicting a decrease in tumor size following the treatment with ML-7, etoposide, and ML-7+etoposide as compared to the tumor removed from an untreated mouse.

FIG. 16B is a bar graph depicting tumor weight from animals treated with ML-7, etoposide, and ML-7+etoposide as compared to control.

FIG. 17 shows a series of photomicrographs depicting increased number of apoptotic cells from rats receiving ML-7 and/or etoposide treatment.

### DETAILED DESCRIPTION OF PREFERRED EMBODIMENTS

It is to be understood that this invention is not limited to the particular methodology, protocols, cell lines, animal species or genera, constructs, or reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein may be used in the practice or testing of the invention, the preferred methods, devices and materials are now described.

The present invention describes methods of using chemical agents to treat cancer. More specifically, the present invention includes methods of using compounds that induce a reduction in myosin light chain phosphorylation to treat cancer. More specifically, the present invention includes methods of using myosin light chain kinase (MLCK) inhibitors to treat cancer. MLCK inhibitors may induce apoptosis in neoplastic cells, prevent proliferation of neoplastic cells, inhibit proliferation of neoplastic cells, and/or prevent or reduce tumor growth in a subject.

In general, as explained above, this invention among other things is a method of use of compounds having certain inhibitory effects, such as MLCK inhibitors, that act through the pathways described herein.

In one embodiment, the present invention is a method of treating a patient having neoplasia. The method includes administering to the patient in need thereof a pharmaceutical composition comprising a therapeutically effective amount of at least one myosin light chain kinase inhibitor in a therapeutically effective amount sufficient to cause a reduction in MLC-P during apoptosis of the neoplastic cells in the patient. The myosin light chain kinase inhibitor may be, including but not limited to, ML-9, ML-7, staurosporine, KT-5926, Calphostin C, H-7, H-8, H-89, HA-1 00, HA-1 077, K-252a, K-252b, Piceatannol, Peptide 18, Sm-I peptide, and Peptide 342-352, all functional equivalents, analogs, conjugates, and pharmaceutically effective derivatives thereof. Preferably, the MLCK inhibitor is ML-7.

In another embodiment, the invention is a method of inducing apoptosis in neoplastic cells in a subject. The method includes administering to the subject a composition comprising at least one myosin light chain kinase inhibitor in a dose effective to induce apoptosis in these neoplastic cells. The myosin light chain kinase inhibitor may be, including but not limited to, ML-9, ML-7, staurosporine, KT-5926, Calphostin C, H-7, H-8, H-89, HA-100, HA-1077, K-252a, K-252b, Piceatannol, Peptide 18, Sm-I peptide, and Peptide 342-352, all functional equivalents, analogs, conjugates, and pharmaceutically effective derivatives thereof. Preferably, the MLCK inhibitor is ML-7.

In another embodiment, the present invention contemplates a method of inducing apoptosis in tumor cells in a subject. The method includes administering to the subject a composition comprising an effective amount of at least one myosin light chain kinase inhibitor and thereby inducing the apoptosis in the tumor cells. The method also comprises evaluating the cells for indication of apoptosis. The method further comprises determining the amount of MLC-P in the tumor cells. The myosin light chain kinase inhibitor may be, including but not limited to, ML-9, ML-7, staurosporine, KT-5926, Calphostin C, H-7, H-8, H-89, HA-100, HA-1 077, K-252a, K-252b, Piceatannol, Peptide 18, Sm-I peptide, and Peptide 342-352, all functional equivalents, analogs, conjugates, and pharmaceutically effective derivatives thereof. Preferably, the MLCK inhibitor is ML-7.

In a further embodiment, the present invention contemplates a method of inhibiting a tumor growth in a subject. The method includes administering to the subject an effective amount of at least one myosin light chain kinase inhibitor in an amount sufficient to cause a reduction in MLC-P during apoptosis of the cells of the tumor, and thereby inhibiting the tumor growth. The myosin light chain kinase inhibitor may be, including but not limited to, ML-9, ML-7, staurosporine, KT-5926, Calphostin C, H-7, H-8, H-89, HA-100, HA-1077, K-252a, K-252b, Piceatannol, Peptide 18, Sm-I peptide, and Peptide 342-352, all functional equivalents, analogs, conjugates, and pharmaceutically effective derivatives thereof. Preferably, the MLCK inhibitor is ML-7.

In yet another embodiment, the present invention contemplates a method of treating a patient having neoplasia. The method includes administering to the patient in need thereof a pharmaceutical composition comprising at least one cytotoxic agent. The method further comprises administering to the patient in need thereof a pharmaceutical composition comprising at least one myosin light chain kinase inhibitor. The compositions are in the amounts, which, together, are effective to cause a reduction in MLC-P during apoptosis of the neoplastic cells in the patient.

An understanding of the mechanisms (e.g., the specific molecular mechanisms) is not necessary to utilize the present invention. While it is not intended that the present invention be limited to any particular mechanism(s), it is believed that the certain compounds, including MLCK inhibitors, used in the methods of the present invention cause a pharmacological perturbation of cellular contractility and perhaps secondarily, cell adhesions, mainly via disruption of the associated cytoskeletal structures or the modulation of their interactions with the membrane and elements of the extracellular matrix. Reduction in contractility and/or perturbation of these adhesions then leads to cell death or apoptosis. It is further believed that certain compounds, including MLCK inhibitors, may be responsible for causing a reduction in phosphorylated form of myosin light chain during apoptosis. Thus, these MLCK inhibitor compounds may be used to induce apoptosis in cells, and more specifically neoplastic cells, in a therapeutically useful manner.

According to the methods of this invention, inhibiting MLCK has the potential for inducing apoptosis in tumor cells. Thus, the invention described herein could have far reaching consequences when considering treatment of the various diseases, such as cancer. Furthermore, the findings described herein suggest a novel target, amenable to genetic or pharmacological manipulation, for treating proliferative disorders.

Definition of Terms

The following definitions are provided in order to aid the reader in understanding the detailed description of the present invention.

The terms “cancer” and “cancerous” refer to or describe the physiological condition in mammals that is typically characterized by unregulated cell growth. The cells that divide and grow uncontrollably invade and disrupt other tissues and spread to other areas of the body (metastasis) through the lymphatic system or the blood stream. Examples of cancer include but are not limited to, carcinoma, lymphoma, blastoma, sarcoma, and leukemia. More particular examples of such cancers include prostate cancer, colon cancer, squamous cell cancer, small-cell lung cancer, non-small cell lunar cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma kidney cancer, liver cancer, vulval cancer, thyroid cancer, hepatic carcinoma and various types of head and neck cancer. Cancer exerts its deleterious effect on the body by 1) destroying the surrounding adjacent tissues: e.g. compressing nerves, eroding blood vessels, or causing perforation of organs; and 2) replacing normal functioning cells in distant sites: e.g. replacing blood forming cells in the bone marrow, replacing bones leading to increased calcium levels in the blood, or in the heart muscles so that the heart fails.

“Tumor”, as used herein, refers to all neoplastic cell growth and proliferation, whether malignant or benign, and all pre-cancerous and cancerous cells and tissues.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, etc.

The term “inhibitor” refers to a molecule which represses or prevents another molecule from engaging in a reaction. More specifically, the term “MLCK inhibitor” refers to a molecule that is able to inhibit MLCK activity and/or expression. The reported MLCK inhibitors include, but are not limited to, ML-9, ML-7, staurosporine, KT-5926, Calphostin C, H-7, H-8, H-89, HA-1 00, HA-1 077, K-252a, K-252b, Piceatannol, Peptide 18, Sm-1 peptide, and Peptide 342-352, all functional equivalents, analogs, conjugates, and pharmaceutically effective derivatives of these MLCK inhibitors. MLCK inhibitors also include an anti-MLCK antibody.

The term “inhibitor analog” refers to a compound that is structurally similar but non-identical to the inhibitor compound. For example, an MLCK inhibitor analog may be a compound that is structurally similar but non-identical to the MLCK inhibitor, such as ML-7. MLCK inhibitor analogs may be branched or un-branched.

The terms “polypeptide”, “peptide”, “protein”, and the like are used interchangeably herein to refer to any polymer of amino acid residues of any length. The polymer can be linear or non-linear (e.g., branched), it may comprise modified amino acids or amino acid analogs, and it may be interrupted by chemical moieties other than amino acids. The terms also encompass an amino acid polymer that has been modified naturally or by intervention; for example, by disulfide bond formation, glycosylation, lipidation, acetylation, phosphorylation, or any other manipulation or modification, such as conjugation with a labeling or bioactive component. According to one embodiment of this invention, MLCK inhibitor may be a peptide. MLCK inhibitor peptides include, but are not limited to, Peptide 18, Sm-I peptide, and Peptide 342-352.

By “conjugation” is meant the process of forming a covalent linkage, with or without an intervening linker, between two moieties, such as an inhibitor analog and a polypeptide. The conjugation can be performed by any method known in the art, such as those described in Wong, Chemistry of protein Conjugation and Cross-linking, 1991, CRC Press, Boca Raton.

The term “conjugate” refers to a compound formed by conjugation process.

The term “functional equivalent” refers to a compound or a peptide or small molecule that is able to function in a similar manner to compounds disclosed herein. For example, a functional equivalent could be compound that is able to function similarly to MLCK inhibitors. For example, the functional equivalent of MLCK inhibitor could bind and interfere with the functioning of MLC and its role in cytoskeleton functioning.

The term “inhibition,” in the context of neoplasia, tumor growth or tumor cell growth, may be assessed by delayed appearance of primary or secondary tumors, slowed development of primary or secondary tumors, decreased occurrence of primary or secondary tumors, slowed or decreased severity of secondary effects of disease, arrested tumor growth and regression of tumors, among others. In the extreme, complete inhibition, is referred to herein as prevention or chemoprevention.

The term “induces cell death” or “capable of inducing cell death” refers to the ability of MLCK inhibitor, alone or in co-treatment with a chemotherapeutic agent to make a viable cell become nonviable. The assay for cell death may be performed using heat inactivated serum (i.e. in the absence of complement) and in the absence of immune effector cells. To determine whether the MLCK inhibitor is able to induce cell death, loss of membrane integrity as evaluated by uptake of propidium iodide (PI), trypan blue (see Moore et al., Cytotechnology 17:1-11, 1995) or 7AAD can be assessed relative to untreated cells.

The phrase “induces apoptosis” or “capable of inducing apoptosis” refers to the ability of MLCK inhibitor, alone or in co-treatment with a chemotherapeutic agent, to induce programmed cell death as determined by binding of Annexin V, fragmentation of DNA, cell shrinkage, dilation of endoplasmic reticulum, cell fragmentation, and/or formation of membrane vesicles (called apoptotic bodies). Various methods are available for evaluating the cellular events associated with apoptosis. For example, phosphatidyl serine (PS) translocation may be measured by annexin binding; DNA fragmentation may be evaluated through DNA laddering as disclosed in the example herein; and nuclear chromatin condensation along with DNA fragmentation may be evaluated by any increase in hypodiploid cells. Preferably, in the context of the present invention, an MLCK inhibitor which induces apoptosis is one which results in about 2 to 50 fold, preferably about 5 to 50 fold, and most preferably about 10 to 50 fold induction of annexin binding relative to untreated cell in an annexin binding assay using cells.

The phrase “therapeutically effective” is intended to qualify the amount of inhibitors in the therapy. This amount will achieve the goal of treating, preventing or inhibiting neoplasia or a neoplasia-related disorder.

The term “prophylactically effective amount” refers to an amount effective in preventing or substantially lessening neoplasia or a neoplasia related disorder.

The phrases “low dose” or “low dose amount”, in characterizing a therapeutically effective amount of the MLCK inhibitor, defines a quantity of such agent, or a range of quantity of such agent, that is capable of improving the neoplastic disease severity while reducing or avoiding one or more antineoplastic-agent-induced side effects, such as myelosupression, cardiac toxicity, alopecia, nausea or vomiting.

The term “pharmaceutically acceptable carrier or adjuvant” refers to a non-toxic carrier or adjuvant that may be administered to a patient, together with a compound of this invention, and which does not destroy the pharmacological activity thereof.

The term “pharmaceutically acceptable derivative” means any pharmaceutically acceptable salt, ester, or salt of such ester, of a compound of this invention or any other compound which, upon administration to a recipient, is capable of providing (directly or indirectly) a compound of this invention or an active metabolite or residue thereof.

Pharmaceutically acceptable salts of the compounds of this invention include, for example, those derived from pharmaceutically acceptable inorganic and organic acids and bases. Examples of suitable acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N— (CA-4 alkyl)salts.

“Therapeutic compound” means a compound useful in the treatment, prevention or inhibition of neoplasia or a neoplasia-related disorder.

A “growth inhibitory agent” when used herein refers to a compound or composition which inhibits growth of a cell, especially cancer cell, either in vitro, ex vivo, or in vivo. Examples of growth inhibitory agents include agents that block cell cycle progression (at a place other than S phase), such as agents that induce GI arrest and M-phase arrest. Classical M-phase blockers include the vincas (vincristine and vinblastine), taxol, and topo II inhibitors such as doxorubicin, epirubicin, daunorubicin, etoposide, and bleomycin. Those agents that arrest GI also spill over into S-phase arrest, for example. DNA alkylating agents such as tamoxifen, prednisone, dacarbazine, mechlorethamine, cisplatin, methotrexate, 5-fluorouracil, and ara-C. Further information may be found in The Molecular Basis of Cancer, Mendelsohn and Israel, eds., Chapter 1, entitled “Cell cycle regulation, oncogenes, and antineoplastic drugs” by Murakami et at., (W B Saunders; Philadelphia, 1995), especially p. 13. Other agents that inhibit growth of cancer cells, as described below include those disclosed herein, including ML-9, ML-7, staurosporine, KT-5926, Calphostin C, H-7, H-8, H-89, HA-1 00, HA-1 077, K-252a, K-252b, Piceatannol, Peptide 18, Sm-I peptide, and Peptide 342-352, all functional equivalents, analogs, conjugates, and pharmaceutically effective derivatives of these MLCK inhibitors.

The term “comprising” means “including the following elements but not excluding others.”

The term “condition” refers to any disease, disorder or effect that produces deleterious biological consequences in a subject.

The term “subject” refers to an animal, or to one or more cells derived from an animal. Preferably, the animal is a mammal, most preferably a human. Cells may be in any form, including but not limited to cells retained in tissue, cell clusters, immortalized cells, transfected or transformed cells, and cells derived from an animal that have been physically or phenotypically altered.

“Mammal” for purposes of treatment refers to any animal classified as a mammal, including humans, domestic and farm animals, and zoo, sports, or pet animals, such as dogs, horses, cats, cattle, pigs, sheep, etc. Preferably, the mammal is human.

The terms “patient,” “subject,” and “recipient” as used in this application refer to any mammal, especially humans.

The Cytoskeleton

The ability of cells to adopt a variety of shapes and carry out coordinated and directed movements depends on complex network of protein filaments that extends throughout the cytoplasm. This network is called the “cytoskeleton.”

The diverse activities of the cytoskeleton depend on three types of protein filaments: actin filaments (actin), microtubules, and intermediate filaments.

Actin is involved in a remarkably wide range of structures, from stiff and relatively permanent extensions of the cell surface to the dynamic three-dimensional networks at the leading edge of a migrating cell. Actin binds a large number of proteins, including those in the myosin family. Muscle myosin belongs to the myosin II subfamily of myosins. Together, actin and myosin II greatly affect the dynamic properties of the cytoskeleton.

A growing body of data suggests that many signaling pathways converge to regulate the actin-myosin 11 interaction in mammalian smooth muscle cells (SMC) and non-muscle cells (NMC). The actin-myosin interaction in these cells is regulated by phosphorylation/dephosphorylation of the 20 kD light chain of myosin 11 (MLC₂₀) by a calcium calmodulin dependent enzyme, myosin light chain kinase (MLCK). Phosphorylation of MLC₂₀ by MLCK stimulates the actin activated, Mg²⁺-dependent ATPase activity of myosin 11 purified from smooth muscle and non-muscle cells (de Lanerolle, P. and Paul, R. J., 1991; Nakano, T. and Hartshorne, D. J., 1995; Somlyo, A. P. and Somlyo, A. V., 1994; Trybus, K. M., 1996; and Kamm, K. E. and Stull, J. T., 2001). To date it has been shown that MLC₂₀ phosphorylation (MLC-P) and dephosphorylation (MLC-DP) is required for smooth muscle contraction and relaxation (reviewed in refs. de Lanerolle, P. and Paul, R. J., 1991; Nakano, T. and Hartshorne, D. J., 1995; and Kamm, K. E. and Stull, J. T., 2001). Other experiments have shown that MLC-P/MLC-DP plays a central role in cell motility (Wilson, A. K., et at., 1991; Wilson, A. K., Takai, A., et al. 1991; and Klemke, R. L., et al., 1997), endothelial cell contractility (Wysolmerski, R. B. and Lagunoff, D., 1990; and Wysolmerski, R. B. and Lagunoff, D., 1991), epithelial barrier function (Hecht, G., et al., 1996; Gandhi, S., et al., 1997; Bergulund, J. J., et al., 2001; and Zolotarevsky, Y., et al., 2002) and secretion from basophilic cells (Choi, O. H., et al., 1994). Furthermore, classic experiments by Mabuchi (Mabuchi, I. and M. Okuno., 1977), Loomis (Knecht, D. A. and W. F. Loomis, 1987) and Spudich (De Lozanne, A. and Spudich, J. A., 1987) have shown that myosin II is essential for cell division in diverse organisms. Yet further studies have shown that MLC-P/MLC-DP plays an important role in cell division in mammalian cells (Sattewhite, L. L., et al., 1992; Fishkind, D. J., et al., 1991; Yamakita, Y., et al, 1994; Totsukawa, G., et al., 2000; Kosako, H., et al., 2000; and Matsumura, F., et al., 2001).

MLCK/MLC-P and GTPase Signaling Pathways

Small G proteins (GTPases) such as rho and Cdc421rac (Vojtek, A. B. and Cooper, J. A., 1995; and Etienne-Manneville, S. and Hall, A., 2002) are also important in regulating cytoskeletal dynamics. Chrzanowska-Wodnicka and Burridge (Chrzanowska-Wodnicka, M. and Burridge, K., 1996) have shown that rho activation stimulates a contractile event that involves MLC-P and results in the formation of stress fibers and focal adhesions in fibroblasts. Activation of rho A by growth factors or phospholipids, was found to increase MLC-P, either by direct phosphorylation of myosin light chains (Amano, M., et al., 1996) and/or by the inactivation of a myosin phosphatase (PP1M) by Rho kinase (Kimura, K., et al., 1996; Uehata, M., et al., 1997). Also, it has also been shown that Rho is required for cell proliferation and migration (Seasholtz, T. M., et al., 2001).

Studies on Erk and PAK provide more support for a direct interaction between GTPases and the MLC-P. The researchers began the studies in this area by investigating the role of MLC-P in ras signaling because ras transformed cells were found to be more motile than nontransformed cells (Kundra, V., et al., 1994) and MLC-P is required for cell motility (Wilson, A. K., et al., 1991). The researchers have found that MLCK is phosphorylated by Erk 1 and 2, in vitro, and that phosphorylation increases MLCK activity at all calmodulin concentrations (Hecht, G., et al., 1996). In addition, expression of a mutationally active MAP kinase (MEK) was found to cause MAP kinase activation, increase MLCK phosphorylation and MLC-P, and enhance cell motility (Klemke, R. L., et al., 1997). Prior to these findings, ras was thought to only stimulate transcription. These experiments, however, identified a novel target for Erk and provided a mechanism by which ras transformation stimulates cell motility.

Furthermore, the researchers investigated rac/MLC-P interactions by studying PAK. PAKs constitute a family of enzymes that is activated when they bind to the activated, GTP-bound form of rac 1 and to Cdc42, but not other GTPases (Sells, M. A. and Chernoff, J., 1997). There are at least 5 separate isoforms of PAK (Sells, M. A. and Chernoff, J., 1997; Jaffer, Z. M. and Chernoff, J., 2002). Although the physiological functions of PAK's are not fully understood, PAK's have been implicated in apoptosis (Rudel, T. and Bokoch, G. M., 1997) and in cancer (Kumar, R. and Vadlamudi, R. K., 2002). Previous experiments have demonstrated that MLCK is phosphorylated by PAK 1 and that this phosphorylation decreases MLCK activity by 50%. Also, it has been shown that cell spreading, the first step in cell motility, is inhibited when MLCK is phosphorylated by PAK 1 (Sanders, L., et al., 1999). Furthermore, it has been reported that PAK 4 regulates cell adhesion and anchorage independent growth (Qu., J., et al., 2001).

Taken together, these data suggest a central role for myosin light chain phosphorylation in regulating cell motility and cell division. However, to date, the details of this involvement are not well understood. Therefore, the numerous experiments described herein were designed to investigate what happens to MLC-P during apoptosis, the opposite of proliferation. Based on previous studies (Mills, J. C., et al., 1998; Sebbagh, M., et al., 2001; Jin, Y., et al., 2001; and Petrache, I., et al., 2003), the researchers suspected that MLC-P would increase during apoptosis because caspases would digest and activate MLCK. The results, described below, suggest that exactly the opposite is true. As described below, the MLC₂₀ are dephosphorylated during apoptosis. Also, without intent to limit the invention, the findings described herein suggest that inhibiting MLCK is sufficient to induce apoptosis and to retard the growth of breast cancer cells in mice, and may be used to treat many types of cancer in humans.

Myosin Light-Chain Kinase Inhibitors

This invention encompasses compounds having certain inhibitory effects to prevent or inhibit tumor growth. Preferably these compounds include myosin light-chain kinase inhibitors.

Several compounds known to be myosin light-chain kinase inhibitors have been identified to be effective in destabilizing cell skeleton forces which then lead to apoptosis in smooth muscle cells in vitro. These compounds primarily inhibit myosin light-chain kinase by inhibiting actomyosin-driven contractility, but other mechanisms of action may also be involved.

Exemplary myosin light chain kinase inhibitors include ML-9, ML-7, staurosporine, Calphostin C, H-7, H-8, H-89, HA-1 00, HA-1 077, K-252a, K-252b, Piceatannol, Peptide 18, Sm-I peptide, Peptide 342-352 (shown in Tables A, B, C), and KT-5926 ([(8R*,9S*,11S*)-(−)₉-hydroxy-9-methoxycarbonyl-8-methyl-14-n-propoxy-2,3,9,10-tetrahydro-8,11-epoxy, 1H,8H, 11H-2,7b,11a-triazadibenzo [a,g]cycloocta [cde]trinden-1-one), BMD (2,3-butanedione 2-monoxime), Fasudil (HA1 077) (Hexahydro-1-(5-isoquinolinesulphonyl)-1H-1,4-diazepine), and W-7 (N-(6-Aminohexyl)-5-chloro-1-naphthalenesulfonamide).

In addition to all MLCK inhibitors depicted in Tables A, B, and C, and those described above, MLCK inhibitors include all functional equivalents, analogs and pharmaceutically effective derivatives of these MLCK inhibitors, which include any pharmaceutically acceptable salt, ester, or salt of such ester, of a compound of this invention or any other compound which, upon administration to a recipient, is capable of providing (directly or indirectly) a compound of this invention or an active metabolite or residue thereof.

The effect of these compounds may involve loosening cell-cell or cell-extracellular matrix adherents junctions of trabecular meshwork cells, or altered cellular contractility, via a direct or indirect effect on actin filaments, actin-myosin interactions, or actin-membrane interactions. It should be noted that described herein are indications that the MLCK inhibitors act due to their effect on myosin light chain kinase. Such inhibition may lead to inhibition of contractility of non-muscle cells or smooth muscle, but will not affect skeletal or cardiac muscle, which uses a different regulatory mechanism.

H-7 is a myosin light-chain kinase and cyclic nucleotide-dependent protein kinase inhibitor. It is believed that H-7's mechanism of action may involve loosening or weakening of cell-cell or cell-extracellular matrix adherents junctions of trabecular meshwork, or altered cellular contractility, via an effect on actin filaments or acto-myosin interactions. Indeed, H-7 has been reported to markedly increase thrombin-induced intercellular gap formation in confluent cultured bovine retinal pigment epithelial cells and induce deterioration of stress fibers in different cultured cells. (Volberg T, et al., 1994). Furthermore, as described herein, there is evidence suggesting that H-7 acts primarily to inhibit myosin light-chain kinase and thus inhibits actomyosin-driven contractility, eventually leading to perturbation of microfilaments and apoptosis. The structure of H-7 is depicted in Table A.

Staurosporine is a microbial alkaloid produced by a Streptomyces species. The compound is reported to possess several biological activities, including antifungal and hypotensive effects, inhibition of platelet aggregation, and promotion of cell differentiation. [T. Tamaoki et al., 1986; H. Matsumoto and Y. Sasaki, 1989).

According to Kaufman, et al. (U.S. Pat. No. 6,110,912), staurosporine is a modulator of serine-threonine (ser-thr) kinases that probably, like H-7, acts through inhibition of myosin light-chain kinase. Its effect on the trabecular meshwork has not been definitively elucidated, though staurosporine might decrease contractility, leading to a total disruption of the microfilament system. Staurosporin includes K-252 (see, for example, Japanese Patent Application No. 62-1 64,626), BMY-41950 (U.S. Pat. No. 5,015,578), UCN-01 (U.S. Pat. No. 4,935,415), TAN-999 (Japanese Patent Application No. 01-1 49,791), TAN-1030A (Japanese Patent Application No. 01-246,288), RK-286C (Japanese Patent Application No. 02-258,724) and functional equivalents and derivatives thereof. Derivatives of staurosporin include those discussed in Japanese Patent Application Nos. 03-72,485; 01-143,877; 02-09,819 and 03-220,194, as well as in PCT International Application Nos. WO 891071 05 and WO 91109034 and European Patent Application Nos. EP 410,389 and EP 296,110. Derivatives of K-252, a natural product, are known. See, for example, Japanese Patent Application Nos. 63-295,988; 62-240,689; 61-268,687; 62-1 55,284; 62-1 55,285; 62-1 20,388 and 63-295,589, as well as PCT International Application No. WO 88107045 and European Patent Application No. EP 323,171. The structures of staurosporine, K-252a, and K-252b are shown in Table B below.

Other MLCK inhibitors contemplated by the present invention for treatment of neoplasia, include, but are not limited to, ML-7, ML-9, and KT5926. It is believed that these compounds will have characteristics similar to H-7 due to their mechanisms of action. (See, e.g., Volberg T, et al., 1994; Nakanishi S, et al., 1990).

ML-7 is a myosin light chain kinase inhibitor commercially available from Sigma or Biomol. Though not as extensively studied as some of the other protein kinase inhibitors, ML-7 has been found to effectively inhibit mouse lung carcinoma 3LL cell attachment to the fibronectin substratum (Isemura M, et al., 1991). ML-7 includes all functional equivalents and derivatives and analogs thereof. The ML-7 structure is depicted in Table A.

TABLE A
Myosin Light Chain Kinase Inhibitors
MLCK Inhibitors

| Inhibitor | | |
|---|---|---|
| NAME | Chemical Name | STRUCTURE |
| A-3,hydrochloride | N-(2-Aminoethyl)-5-chloronaphthalene-1-sulfonamide, HCl | 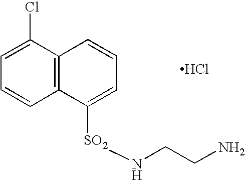 |
| Calphostin C(UCN-1028C)(Alexis) | | 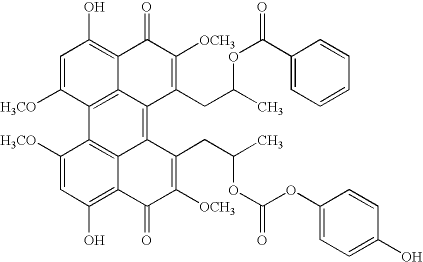 |
| H-7,dihydrochloride | 1-(5-isoquinolinyl-sulfonyl)-2-methylpiperazine | 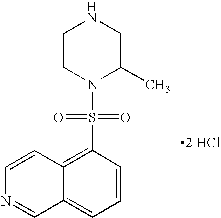 |
| H-8,dihydrochloride(Alexis) | N-[2-Methylamino)ethyl]-5-isoquinolinesulfonamide•2HCl | 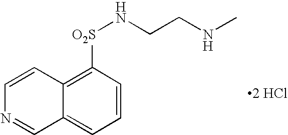 |
| H-89,dihydrochloride(Alexis) | N-[2-p-Bromocinnamylamino)ethyl]-5-isoquinolinesulfonamide•2HCl | 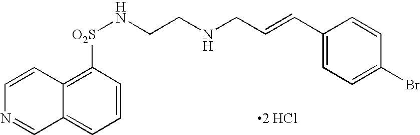 |
| HA-100,dihydrochloride(Alexis) | 1-(5-Isoquinolinylsulfonyl)piperazine•2HCl | 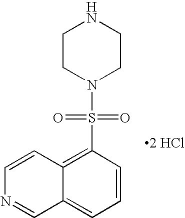 |
| HA-1077,dihydrochloride(Alexis) | 1-(5-Isoquinolinylsulfonyl)homopiperazine•2HCl | 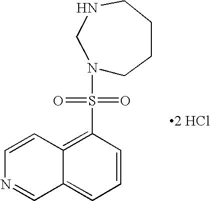 |
| ML-7,hydrochloride(Biomol) | 1-(5-Iodonaphthalene-1-sulfonyl)-1H-hexahydro-1,4-diazepine•HCl | 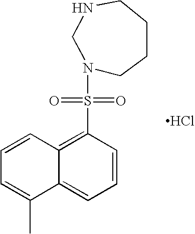 |
| ML-9,hydrochloride(Biomol) | 1-(5-Chloronaphthalene-1-sulfonyl)-1H-hexahydro-1,4-diazepine•HCl | 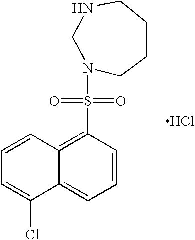 |
| Piceatannol(EMDBiosciences) | trans-3,3′,4,5′-Tetrahydroxystilbene | 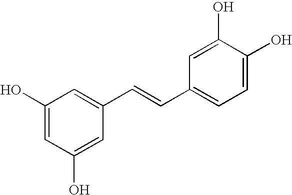 |

ML-9 is also a myosin light chain kinase inhibitor and it is also commercially available from Sigma or Biomol. ML-9 also includes all functional equivalents and derivatives and analogs thereof. The ML-9 structure is depicted in Table A.

KT5926 is a potent and selective inhibitor of myosin light-chain kinase. The compound has been shown to inhibit both Ca²⁺/calmodulin-dependent and -independent smooth muscle myosin light chain kinases as well. Though KT5926 is deemed to be a selective inhibitor of myosin light chain kinase, it, too inhibits other protein kinases, e.g., CAMP-dependent protein kinase, but with relatively high Ki values. The chemical structure of KT5926 is depicted in Table B above (Nakanishi S, et al., 1990).

In addition, MLCK inhibitors include commercially available polypeptides having amino acid sequences listed in Table C below.

TABLE B
Structures of Staurosporines

| | |
|---|---|
| Staurosporine(EMD Biosciences) | 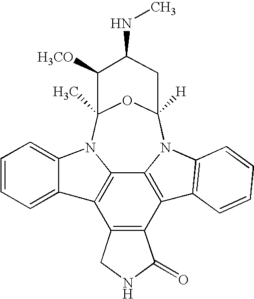 |
| K-252a (Calbiochem) | 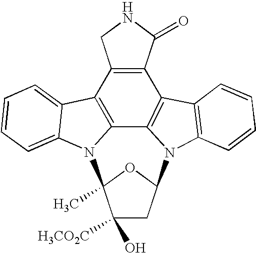 |
| K-252b (Calbiochem) | 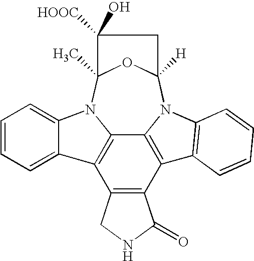 |
| KT-5926 | 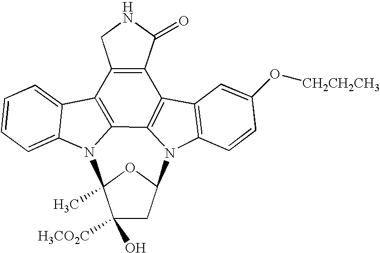 |

TABLE C
MLCK Inhibitor Polypeptides

| | |
|---|---|
| SM-1 (MLCK Inhibitor | H-AKKLSKDRMKKYMARRKWQKTG-NH₂ |
| Peptide 480-501) | (SEQ ID NO: 1) |
| (Calbiochem) | |
| MLCK Inhibitor Pep- | H-RKKYKYRRK-NH₂ |
| tide 18 (Calbiochem) | (SEQ ID NO: 2) |
| MLCK Inhibitor Pep- | Lys-Arg-Arg-Trp-Lys-Lys-Asn- |
| tide 342-352 (Biomol) | Phe-Ile-Ala-Val-NH₂ |
| | (SEQ ID NO: 3) |

In yet another embodiment, MLCK inhibitors may include an anti-MLCK antibody. The term “antibody” refers to a monoclonal or a polyclonal antibody per se, immunologically effective fragments thereof (e.g., Fab, Fab′, or F(ab′)₂), or a single chain version of the antibodies, usually designated as Fv regions. Methods of producing polyclonal and monoclonal antibodies, including binding fragments and single chain versions are well known in the art.

The anti-MLCK antibody may be, for example, one as previously described by de Lanerolle, et al. 1981. To develop the anti-MLCK antibody, de Lanerolle et al. has used an immunological approach to first localize myosin light chain kinase in non-muscle cells. MLCK was then purified from turkey gizzard smooth muscle and the antibodies to this protein were raised in rabbits. When these antibodies were tested for their ability to inhibit the activity of purified MLCK, de Lanerolle et al. found that fifty percent of the MLCK activity was inhibited in the presence of about 8 pmol of antibody (de Lanerrolle, et al., 1981). There was also a significant reduction in the rate of phosphorylation when the MLCK was incubated with the anti-MLCK antibody developed by de Lanerrolle et al. (de Lanerolle et al., 1981).

Therapy

The methods of the present invention are useful for the treatment, prevention or inhibition of neoplasia or a neoplasia-related disorder including malignant tumor growth, benign tumor growth and metastasis.

Malignant tumor growth locations comprise the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, dermis, and head and neck region.

More specific locations within the above systems include the following. Malignant tumor growth locations in the nervous system comprise the brain and spine. Malignant tumor growth locations in the respiratory tract system comprise the lung and bronchus. Malignant tumor growths in the lymphatic system comprise Hodgkin's lymphoma and non-Hodgkin's lymphoma. Malignant tumor growth locations in the hepatic system comprise the liver and intrahepatic bile duct. Malignant tumor growth locations in the musculoskeletal system comprise bone, bone marrow, joint, muscle, and connective tissue. Malignant tumor growth locations in the digestive tract comprise the colon, small intestine, large intestine, stomach, colorectal, pancreas, liver, and rectum. Malignant tumor growth locations in the renal system comprise the kidney and renal pelvis. Malignant tumor growth locations in the male reproductive system comprise the prostate, penis and testicle. Malignant tumor growth locations in the female reproductive system comprise the ovary and cervix. Malignant tumor growth locations in the urinary tract comprise the bladder, urethra, and ureter. Malignant tumor growth locations in the nasal system comprise the nasal tract and sinuses. Malignant tumor growth locations in the gastrointestinal tract comprise the esophagus, gastric fundus, gastric antrum, duodenum, hepatobiliary, ileum, jejunum, colon, and rectum. Malignant tumor growth in the dermis comprises melanoma and basal cell carcinoma. Malignant tumor growth locations in the head and neck region comprise the mouth, pharynx, larynx, thyroid, and pituitary. Malignant tumor growth locations further comprise smooth muscle, striated muscle, and connective tissue. Malignant tumor growth locations even further comprise endothelial cells and epithelial cells. Malignant tumor growth may be breast cancer. Malignant tumor growth may be in soft tissue. Malignant tumor growth may be a viral-related cancer, including cervical, T-cell leukemia, lymphoma, and Kaposi's sarcoma. Benign tumor growth locations comprise the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, dermis, and head and neck region.

More specific locations for the above systems include the following locations. Benign tumor growth locations in the nervous system comprise the brain and spine. Benign tumor growth locations in the respiratory tract system comprise the lung and bronchus. A benign tumor growth in the lymphatic system may comprise a cyst. Benign tumor growth locations in the hepatic system comprise the liver and intrahepatic bile duct. Benign tumor growth locations in the musculoskeletal system comprise bone, bone marrow, joint, muscle, and connective tissue. Benign tumor growth locations in the digestive tract comprise the colon, small intestine, large intestine, stomach, colorectal, pancreas, liver, and rectum. A benign tumor growth in the digestive tract may comprise a polyp. Benign tumor growth locations in the renal system comprise the kidney and renal pelvis. Benign tumor growth locations in the male reproductive system comprise the prostate, penis and testicle. Benign tumor growth in the female reproductive system may comprise the ovary and cervix. Benign tumor growth in the female reproductive system may comprise a fibroid tumor, endometriosis or a cyst. Benign tumor growth in the male reproductive system may comprise benign prostatic hypertrophy (BPH) or prostatic intraepithelial neoplasia (PIN). Benign tumor growth locations in the urinary tract comprise the bladder, urethra, and ureter. Benign tumor growth locations in the nasal system comprise the nasal tract and sinuses. Benign tumor growth locations in the gastrointestinal tract comprise the esophagus, gastric fundus, gastric antrum, duodenum, hepatobiliary, ileum, jejunum, colon, and rectum. Benign tumor growth locations in the head and neck region comprise the mouth, pharynx, larynx, thyroid, and pituitary. Benign tumor growth locations further comprise smooth muscle, striated muscle, and connective tissue. Benign tumor growth locations even further comprise endothelial cells and epithelial cells. Benign tumor growth may be located in the breast and may be a cyst or fibrocystic disease. Benign tumor growth may be in soft tissue.

Metastasis may be from a known primary tumor site or from an unknown primary tumor site. Metastasis may be from locations comprising the nervous system, cardiovascular system, circulatory system, respiratory tract, lymphatic system, hepatic system, musculoskeletal system, digestive tract, renal system, male reproductive system, female reproductive system, urinary tract, nasal system, gastrointestinal tract, dermis, and head and neck region. Metastasis from the nervous system may be from the brain, spine, or spinal cord. Metastasis from the circulatory system may be from the blood or heart. Metastasis from the respiratory system may be from the lung or broncus. Metastasis from the lymphatic system may be from a lymph node, lymphoma, Hodgkin's lymphoma or non-Hodgkin's lymphoma. Metastasis from the hepatic system may be from the liver or intrahepatic bile duct. Metastasis from the musculoskeletal system may be from locations comprising the bone, bone marrow, joint, muscle, and connective tissue. Metastasis from the digestive tract may be from locations comprising the colon, small intestine, large intestine, stomach, colorectal, pancreas, gallbladder, liver, and rectum. Metastasis from the renal system may be from the kidney or renal pelvis. Metastasis from the male reproductive system may be from the prostate, penis or testicle. Metastasis from the female reproductive system may be from the ovary or cervix. Metastasis from the urinary tract may be from the bladder, urethra, or ureter. Metastasis from the gastrointestinal tract may be from locations comprising the esophagus, esophagus (Barrett's), gastric fundus, gastric antrum, duodenum, hepatobiliary, ileum, jejunum, colon, and rectum. Metastasis from the dermis may be from a melanoma or a basal cell carcinoma. Metastasis from the head and neck region may be from locations comprising the mouth, pharynx, larynx, thyroid, and pituitary. Metastasis may be from locations comprising smooth muscle, striated muscle, and connective tissue. Metastasis may be from endothelial cells or epithelial cells. Metastasis may be from breast cancer. Metastasis may be from soft tissue. Metastasis may be from a viral-related cancer, including cervical, T cell leukemia, lymphoma, or Kaposi's sarcoma. Metastasis may be from tumors comprising a carcinoid tumor, gastrinoma, sarcoma, adenoma, lipoma, myoma, blastoma, carcinoma, fibroma, or adenosarcoma.

Malignant or benign tumor growth may be in locations comprising the genital system, digestive system, breast, respiratory system, urinary system, lymphatic system, skin, circulatory system, oral cavity and pharynx, endocrine system, brain and nervous system, bones and joints, soft tissue, and eye and orbit.

Metastasis may be from locations comprising the genital system, digestive system, breast, respiratory system, urinary system, lymphatic system, skin, circulatory system, oral cavity and pharynx, endocrine system, brain and nervous system, bones and joints, soft tissue, and eye and orbit.

The methods and compositions of the present invention may be used for the treatment, prevention or inhibition of neoplasia or neoplasia-related disorders.

Such disorders include acral lentiginous melanoma, actinic keratoses, acute lymphocytic leukemia, acute myeloid leukemia, adenocarcinoma, adenoid cycstic carcinoma, adenomas, adenosarcoma, adenosquamous carcinoma, anal canal cancer, anal cancer, anorectum cancer, astrocytic tumors, bartholin gland carcinoma, basal cell carcinoma, benign cysts, biliary cancer, bone cancer, bone marrow cancer, brain cancer, breast cancer, bronchial cancer, bronchial gland carcinomas, carcinoids, carcinoma, carcinosarcoma, cholangiocarcinoma, chondosarcoma, choriod plexus papilloma/carcinoma, chronic lymphocytic leukemia, chronic myeloid leukemia, clear cell carcinoma, colon cancer, colorectal cancer, connective tissue cancer, cystadenoma, cysts of the female reproductive system, digestive system cancer, digestive tract polyps, duodenum cancer, endocrine system cancer, endodermal sinus tumor, endometrial hyperplasia, endometrial stromal sarcoma, endometrioid adenocarcinoma, endometriosos, endothelial cell cancer, ependymal cancer, epithelial cell cancer, esophagus cancer, Ewing's sarcoma, eye and orbit cancer, female genital cancer, fibroid tumors, focal nodular hyperplasia, gallbladder cancer, gastric antrum cancer, gastric fundus cancer, gastrinoma, germ cell tumors, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, kidney and renal pelvic cancer, large cell carcinoma, large intestine cancer, larynx cancer, leiomyosarcoma, lentigo maligna melanomas, leukemia, liver cancer, lung cancer, lymphoma, male genital cancer, malignant melanoma, malignant mesothelial tumors, medulloblastoma, medulloepithelioma, melanoma, meningeal cancer, mesothelial cancer, metastatic carcinoma, mouth cancer, mucoepidermoid carcinoma, multiple myeloma, muscle cancer, nasal tract cancer, nervous system cancer, neuroblastoma, neuroepithelial adenocarcinoma nodular melanoma, non-epithelial skin cancer, non-Hodgkin's lymphoma, oat cell carcinoma, oligodendroglial cancer, oral cavity cancer, osteosarcoma, ovarian cancer, pancreatic cancer, papillary serous adenocarcinoma, penile cancer, pharynx cancer, pituitary tumors, plasmacytoma, prostate cancer, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous carcinoma, squamous cell carcinoma, stomach cancer, striated muscle cancer, submesothelial cancer, superficial spreading melanoma, T cell leukemia, testis cancer, thyroid cancer, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, vipoma, vulva cancer, well differentiated carcinoma, and Wilm's tumor.

Preferably, tumors that are most sensitive to treatment with certain compounds of this invention, including the MLCK inhibitors, include solid tumors because these tumors are connected to the extracellular matrix and have well-organized cytoskeletons. Examples of solid tumors, which are tumors of body tissues other than blood, bone marrow, or the lymphatic system, include, but are not limited to colon carcinoma, prostate cancer, breast cancer, lung cancer, skin cancer, liver cancer, bone cancer, ovarian cancer, pancreatic cancer, brain cancer, head and neck cancer, lymphoma and other solid tumors.

In a preferred embodiment, tumors that are most sensitive to treatment with the MLCK inhibitors include breast and prostate cancers.

Combination Therapy

In another aspect, the invention includes methods for inhibiting the proliferation, and/or enhancing reduction of, neoplastic cells, by contacting the cells with at least one anti-cancer drug, i.e. chemotherapeutic, cytotoxic agent, and a composition comprising an agent capable of causing a reduction in MLC-P during apoptosis. Preferably, the agent capable of causing the reduction in MLC-P during apoptosis is an MLCK inhibitor compound.

In another aspect, the invention includes methods for inhibiting the proliferation, and/or enhancing reduction of, neoplastic cells, by contacting the cells with at least one anti-cancer drug, i.e. chemotherapeutic, cytotoxic agent, and at least one MLCK inhibitor. In general, the method includes the step of contacting pathological neoplastic cells with an amount of at least one cytotoxic agent and at least one MLCK inhibitor, which in combination, is effective to cause a reduction in MLC-P and reduce or inhibit the proliferation of the cell, or induce cell death, i.e. apoptosis. The present method may be performed on cells in culture, e.g., in vitro or ex vivo, or may be performed on cells present in a subject, e.g. as part of an in vivo therapeutic protocol. The therapeutic regimen can be carried out on a human or on other animal subjects. The enhanced therapeutic effectiveness of the combination therapy of the present invention represents a promising alternative to conventional highly toxic regimens of anti-cancer agents.

While the MLCK inhibitors may be utilized alone, the subject methods are preferably combined with other anticancer agents, e.g., antimicrotubule agents, topoisomerase I inhibitors, topoisomerase II inhibitors, antimetabolites, mitotic inhibitors, alkylating agents, intercalating agents, agents capable of interfering with a signal transduction pathway (e.g., a protein kinase C inhibitor, e.g., an anti-hormone, e.g., an antibody), agents that promotes apoptosis and/or necrosis, an interferon, an interleukin, a tumor necrosis factor, and radiation. Specific anticancer agents include paclitaxel, interferon alpha, gemcitabine, fludarabine, irinotecan, carboplatin, cisplatin, taxotere, doxorubicin, epirubicin, 5-fluorouracil, UFT, tamoxifen, goserelin, ketoconazole, Herceptin, anti-CD20, leuprolide (Lupron) and flutamide). Co-administration with MLCK inhibitors may improve, enhance, or potentate the efficacy of these anticancer agents. This may also allow for the administration of lower doses of these anticancer agents, thus reducing the induction of side effects in a subject.

The subject method may be useful in treating malignancies of the various organ systems, such as those affecting lung, breast, lymphoid, gastrointestinal (e.g., colon), and genitourinary tract (e.g., prostate), pharynx, as well as adenocarcinomas which include malignancies such as most colon cancer, rectal cancer, renal cell carcinoma, prostate cancer and/or testicular tumors, non-small cell carcinoma of the lung, cancer of the small intestine and cancer of the esophagus. Exemplary solid tumors that may be treated include: pancreatic cancer, breast cancer, ovarian cancer, prostate cancer, and others previously mentioned above.

The MLCK inhibitor is preferably administered in combination with at least one cytotoxic agent. The term “in combination” in this context means that the agents are given substantially contemporaneously, either simultaneously or sequentially. If given sequentially, at the onset of administration of the second compound, the first of the two compounds is preferably still detectable at effective concentrations at the site where treatment effect is desired.

For example, the MLCK inhibitors may be used in combination therapy with conventional cancer chemotherapeutics. Conventional treatment regimens for leukemia and for other tumors include radiation, antitumor agents, interferons, interleukins, tumor necrosis factors, or a combination of two or more of these agents.

For example, the subject method may involve, in addition to the use of at least one MLCK inhibitor, one or more other antitumor agents, propargyl-5,8 dideazafolate (CB3717), 10-ethyl-I10-deaza-aminopterin, deoxycytidine, 5-aza-cytosine arabinoside, N-4-palmitoyl-ara C, 2′-azido-2′-deoxy-ara C, N4-behenoyl-ara C, CCNU (lomustine), estramustine, MeCCNU, triethylene melamine, trenimon, dimethyl busulfan, streptozotocin, chlorozotocin, procarbazine, hexamethylmelamine (Altretamine pentamethylmelamine (PMM), tetraplatin, oxaliplatin, platinum-DACH, aziridinylbenzoquinone (AZQ), bleomycin, tallysomycin S₁₀^{b}, liblomycin, pepleomycin, asparaginase (Elspar), pegaspargase (Oncaspar), Cladrbine (leustatin), porfimer sodium (Photofrin), amonofide, deoxyspergualin, dihydrolenperone, flavone acetic acid, gallium nitrate, and hexamethylene bisacetamine (HMBA).

Particular combinations of several cytotoxic agents may be used depending on the type of hyperproliferative disorder to be treated. For example, in lung cancer, a combination of paclitaxel and carboplatin, or a combination of gemcitabine and cisplatin is used. In hormone refractory prostate cancer, a combination of estramustine phosphate and taxotere or a combination of doxorubicin and ketoconazole is used. For metastatic breast cancer, a combination of cyclophosphamide, doxorubicin and 5-fluorouracil is used. For advanced breast cancer that overexpresses the HER21neu oncogene, a combination of an anti-Her2lneu antibody (e.g., Herceptin) and cisplatin is used. For advanced or metastatic colorectal cancer, a combination of 5-fluorouracil and leucovorin is used. All of the conventional anticancer drugs are highly toxic and tend to make patients quite ill while undergoing treatment; vigorous therapy is based on the premise that unless every cancer cell is destroyed, the residual cells will multiply and cause a relapse. Cytotoxic agents are also used to treat benign hyperplasia disorders. For example, psoriasis is treated with 5-fluorouracil.

Treatment may be initiated with smaller dosages that are less than the optimum dose of the agent. Thereafter, the dosage should be increased by small increments until the optimum effect under the circumstances is reached. For convenience, the total daily dosage may be divided and administered in portions during the day if desired. A therapeutically effective antitumor amount and a prophylactically effective antitumor amount of an MLCK inhibitor or a cytotoxic agent is expected to vary from about 0.1 milligram per kilogram of body weight per day (mg/kg/day) to about 100 mg/kg/day.

Compounds which are determined to be effective for the prevention or treatment of tumors or for the prevention or treatment of benign hyperproliferative disorders in animals, e.g., dogs, rodents, may also be useful in treatment of tumors in humans. Those skilled in the art of treating tumor in humans will know, based upon the data obtained in animal studies, the dosage and route of administration of the compound to humans. In general, the dosage and route of administration in humans is expected to be similar to that in animals, when adjusted for body surface area.

Determination of a therapeutically effective anti-tumor amount and a prophylactically effective antitumor amount of a MLCK inhibitor and cytotoxic agent may be readily made by the physician or veterinarian (the “attending clinician”). The dosages may be varied depending upon the requirements of the patient, the severity of the condition being treated and the particular agent being employed. In determining the dose, a number of factors are considered by the attending clinician, including, but not limited to: the specific hyperplastic/neoplastic cell involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment; and other relevant circumstances. U.S. Pat. No. 5,427,916, for example, describes method for predicting the effectiveness of anti-neoplastic therapy in individual patients.

Compositions and Administration of MLCK Inhibitor Compounds and Cytotoxic Compounds

As described above the present invention contemplates using certain compounds, such as MLCK inhibitors, in therapeutic compositions, either alone or in combination with other antitumor agents. These compositions mainly disrupt the cytoskeletal structures and their interactions with the underlying membrane and/or the extracellular matrix to cause a reduction in MLC-P and induce apoptosis in the neoplastic cells. It is not intended that the present invention be limited by the particular nature of the therapeutic preparation.

MLCK inhibitor compounds of this invention may be formulated into pharmaceutical compositions together with pharmaceutically acceptable carriers for oral administration in solid or liquid form, or for rectal or topical administration, although carriers for oral administration are most preferred.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids, antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming, counterions such as sodium; and/or nonionic surfactants such as TWEEN, polyethylene glycol (PEG), and PLURONICS.

Pharmaceutically acceptable carriers for oral administration include capsules, tablets, pills, powders, troches and granules. In such solid dosage forms, the carrier may comprise at least one inert diluent such as sucrose, lactose or starch. Such carriers may also comprise, as is normal practice, additional substances other than diluents, e.g., lubricating agents such as magnesium stearate. In the case of capsules, tablets, troches and pills, the carriers may also comprise buffering agents. Carriers such as tablets, pills and granules may be prepared with enteric coatings on the surfaces of the tablets, pills or granules. Alternatively, the enterically coated compound may be pressed into a tablet, pill, or granule, and the tablet, pill or granules for administration to the patient. Preferred enteric coatings include those that dissolve or disintegrate at colonic pH such as shellac or Eudraget S.

Pharmaceutically acceptable carriers include liquid dosage forms for oral administration, e.g., pharmaceutically acceptable emulsions, solutions, suspensions, syrups and elixirs containing inert diluents commonly used in the art, such as water. Besides such inert diluents, compositions may also include adjuvants such as wetting agents, emulsifying and suspending agents, and sweetening, flavoring and perfuming agents.

Pharmaceutically acceptable carriers for topical administration include DMSO, alcohol or propylene glycol and the like that may be employed with patches or other liquid-retaining material to hold the medicament in place on the skin so that the medicament will not dry out.

Pharmaceutically acceptable carriers for rectal administration are preferably suppositories that may contain, in addition to the compounds of the invention, excipients such as cocoa buffer or a suppository wax, or gel.

The pharmaceutically acceptable carrier and compounds of this invention are formulated into unit dosage forms for administration to a patient. The dosage levels of active ingredient (i.e., compounds of this invention) in the unit dosage may be varied so as to obtain an amount of active ingredient effective to achieve lesion-eliminating activity in accordance with the desired method of administration (i.e., oral or rectal). The selected dosage level therefore depends upon the nature of the active compound administered, the route of administration, the desired duration of treatment, and other factors. If desired, the unit dosage may be such that the daily requirement for active compound is in one dose, or divided among multiple doses for administration, e.g., two to four times per day.

The compounds of this invention may be formulated with pharmaceutically acceptable carriers into unit dosage forms in a conventional manner so that the patient in need of therapy can periodically (e.g., once or more per day) take a compound according to the methods of this invention. The exact initial dose of the compounds of this invention can be determined with reasonable experimentation. In general, the initial dosage calculation would also take into consideration several factors, such as the formulation and mode of administration, e.g. oral or intravenous, of the particular compound. A total daily oral dosage of about 0.1 milligram (mg) to about 2.0 gram (gr) of such compounds would achieve a desired systemic circulatory concentration. More preferably, as discussed above, an oral dose from about 0.1 mg per kilogram of body weight per day (mg/kg/day) to about 100 mg/kg/day is most preferred and appropriate in humans.

These therapeutic formulations may be administered to mammals for veterinary use, such as with domestic animals, and clinical use in humans in a manner similar to other therapeutic agents.

Preferably, the treatment of mammalian cells in need of MLCK inhibition with a compound of this invention should be continuous over an extended period of time. By continuous, it is not meant to suggest that drug be present or taken all the time. It means that the drug is present most of the time at levels sufficient to cause neoplastic cell death.

The pharmaceutical compositions of this invention are preferably packaged in a container (e.g., a box or bottle, or both) with suitable printed material (e.g., a package insert) containing indications and directions for use in the treatment of a disease where MLCK inhibition is desired, etc.

Furthermore, the MLCK inhibitors may be administered via intravenous infusion, intravenous bolus, subcutaneously, and front loaded.

The term “intravenous infusion” refers to introduction of a drug into the vein of an animal or human patient over a period of time greater than approximately 5 minutes, preferably between approximately 30 to 90 minutes, although, according to the invention, intravenous infusion is alternatively administered for 10 hours or less.

The term “intravenous bolus” or “intravenous push” refers to drug administration into a vein of an animal or human such that the body receives the drug in approximately 15 minutes or less, preferably 5 minutes or less.

The term “subcutaneous administration” refers to introduction of a drug under the skin of an animal or human patient, preferable within a pocket between the skin and underlying tissue, by relatively slow, sustained delivery from a drug receptacle. The pocket may be created by pinching or drawing the skin up and away from underlying tissue.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug deliver, pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is preferably less than approximately 15 minutes, more preferably less than 5 minutes, and most preferably less than 60 seconds. Administration is preferably within a pocket between the skin and underlying tissue, where the pocket is created, for example, by pinching or drawing the skin up and away from underlying tissue.

The term “front loading,” when referring to drug administration is meant to describe an initially higher dose followed by the same or lower doses at intervals. The initial higher dose or doses are meant to more rapidly increase the animal or human patient's serum drug concentration to an efficacious target serum concentration.

One mode of administration for the MLCK inhibitor compounds entails oral administration.

Another mode of administration of the MLCK inhibitor entails intravenous administration.

Evaluation of the Effectiveness of the MLCK Inhibitor Therapy

A. Determining Whether a Compound Reduces Tumor Cell Growth

In one embodiment, the method of the present invention involves determining whether a compound reduces the growth of tumor cells. Various cell lines could be used in the sample depending on the tissue to be tested. For example, these cell lines include: Mm5MT murine mammary tumor; K1735 melanoma; and MatLyLu prostate cancer cells (MLL). Additional cell lines include SW-480-colonic adenocarcinoma; HT-29-colonic adenocarcinoma, A-427-lung adenocarcinoma carcinoma; MCF-7-breast adenocarcinoma; UACC-375-melanoma line; and DU145-prostrate carcinoma. These cell lines are well characterized and they are among cell lines used by the United States National Cancer Institute in their screening program for new anti-cancer drugs. Cytotoxicity data obtained using these cell lines are indicative of an inhibitory effect on neoplastic lesions.

For example, compound's ability to inhibit tumor cell growth can be measured using the HT-29 human colon carcinoma cell line obtained from ATCC. HT-29 cells have previously been characterized as a relevant colon tumor cell culture model (Fogh J, and Trempe G, 1975). HT-29 cells are maintained in RPMI media supplemented with 5% fetal bovine calf serum (Gemini Bioproducts, Inc., Carlsbad, Calif.) and 2 mm glutamine, and 1% antibiotic-antimycotic in a humidified atmosphere of 95% air and 5% CO₂ at 37° C. Briefly, HT-29 cells are plated at a density of 500 cells/well in 96 well microtiter plates and incubated for 24 hours at 37° C. prior to the addition of compound. Each determination of cell number involved six replicates. After six days in culture, the cells are fixed by the addition of cold trichloroacetic acid to a final concentration of 10% and protein levels are measured using the sulforhodamine B (SRB) calorimetric protein stain assay as previously described by Skehan P, et al., 1990, which is incorporated herein by reference.

In addition to the SRB assay, a number of other methods are available to measure growth inhibition and could be substituted for the SRB assay. These methods include counting viable cells following trypan blue staining, labeling cells capable of DNA synthesis with BrdU or radiolabeled thymidine, neutral red staining of viable cells, or MTT staining of viable cells.

Significant tumor cell growth inhibition greater than about 50% at a dose of 20 μM or below is further indicative that the compound is useful for treating neoplastic lesions. Preferably, an ICS0 value is determined and used for comparative purposes. This value is the concentration of drug needed to inhibit tumor cell growth by 50% relative to the control. Preferably, the IC50 value should be less than 30 μM for the compound to be considered further for potential use for treating neoplastic lesions.

Furthermore, in another embodiment of this invention, the determination whether a compound, more specifically an MLCK inhibitor is able to reduce the growth of tumor cells is by implanting tumor cells into an animal and treating the animal with an MLCK inhibitor compound. For example, Mm5MT cells can be injected into an animal. Mm5MT cell line was obtained from ATCC. Once the tumors are established, the animals may be treated with compositions including MLCK inhibitors at varying concentrations for 4 weeks. 4 weeks post injection with the tumor cells, the animals are sacrificed, tumors removed, and then examined using histological and physiological evaluation methods.

B. Determining Whether a Compound Induces Apoptosis

In another embodiment, the method of inducing apoptosis in neoplastic cells includes administering to the subject in need of such administration an effective amount of a composition comprising at least one MLCK inhibitor and thereby inducing the apoptosis in tumor cells. The method of the present invention further involves evaluating whether the compound induces apoptosis in cultures of tumor cells.

Two distinct forms of cell death may be described by morphological and biochemical criteria: necrosis and apoptosis. Necrosis is accompanied by increased permeability of the plasma membrane; the cells swell and the plasma membrane ruptures within minutes. Apoptosis is characterized by membrane bleeding, condensation of cytoplasm and the activation of endogenous endonucleases.

Apoptosis occurs naturally during normal tissue turnover and during embryonic development of organs and limbs. Apoptosis also is induced by cytotoxic T-lymphocytes and natural killer cells, by ionizing radiation and by certain chemotherapeutic drugs. Inappropriate regulation of apoptosis is thought to play an important role in many pathological conditions including cancer, AIDS, or Alzheimer's disease, etc.

Tumors obtained from the mice that were treated with DMSO or a composition comprising an MLCK inhibitor may be processed for histological studies. For example, sections can be prepared from tumors removed from mice receiving DMSO or ML-7, or other MLCK inhibitor compounds and stained with TUNEL reagent (to identify apoptotic cells) and DAPI (to visualize nuclei). The sections can be fixed in 4% paraformaldehyde1PBS and permeabilized in a buffer containing 0.1% sodium citrate and 0.1% Triton X-100 in PBS for 2 min on ice. Permeabilized tissue will be stained with FITC labeled terminal deoxynucleotidyl transferase dUTP nick-end labeling (TUNEL) enzyme reagent using the In Situ Cell Death Detection Kit (Roche Molecular Biochemicals, Indianapolis, Ind.) as described by the manufacturer. Cover slips will be mounted using Vectashield containing DAPI and examined using a Zeiss LSM 510 Laser confocal microscope.

Also, compounds may be screened for induction of apoptosis using cultures of tumor cells maintained under conditions as described above. Treatment of cells with test compounds involves either pre- or post-confluent cultures and treatment for two to seven days at various concentrations. Apoptotic cells are measured in both the attached and “floating” compartments of the cultures. Both compartments are collected by removing the supernatant, trypsinizing the attached cells, and combining both preparations following a centrifugation wash step (5 minutes at 150× G). The protocol for treating tumor cell cultures with ML-7 and related compounds to obtain a significant amount of apoptosis has been described in the examples. In short, the protocol includes collecting both floating and attached cells, identification of the optimal treatment times and dose range for observing apoptosis, and identification of optimal cell culture conditions.

Following treatment with a composition comprising an MLCK inhibitor compound, cultures may be assayed for apoptosis and necrosis by florescent microscopy following labeling with acridine orange and ethidium bromide. The method for measuring apoptotic cell number has previously been described by Duke & Cohen, “Morphological And Biochemical Assays Of Apoptosis,” Current Protocols In Immunology, Coligan et al., eds., 3.1 7.1-3.17.16 (1992), which is incorporated herein by reference.

For example, floating and attached cells can be collected by trypsinization and washed three times in PBS. Aliquots of cells can be centrifuged, the pellet re-suspended in media and a dye mixture containing acridine orange and ethidium bromide prepared in PBS, and mixed gently. The mixture can then be placed on a microscope slide and examined for morphological features of apoptosis.

Apoptosis may also be quantified by measuring an increase in DNA fragmentation in cells that have been treated with test compounds. Commercial photometric EIA for the quantitative, in vitro determination of cytoplasmic histone-associated-DNA-fragments (mono- and oligonucleosomes) are available (Cell Death Detection ELISA^{okys} Cat. No. 1,774,425, Boehringer Mannheim). The Boehringer Mannheim assay is based on a sandwich-enzyme-immunoassay principle using mouse monoclonal antibodies directed against DNA and histones, respectively. This allows the specific determination of mono- and oligonucleosomes in the cytoplasmic fraction of cell lysates.

According to the vendor, apoptosis is measured in the following fashion. The sample (cell-lysate) is placed into a streptavidin-coated microtiter plate (“MTP”). Subsequently, a mixture of anti-histone biotin and anti-DNA peroxidase conjugate are added and incubated for two hours. During the incubation period, the anti-histone antibody binds to the histone-component of the nucleosomes and simultaneously fixes the immunocomplex to the streptavidin-coated MTP via its biotinylation. Additionally, the anti-DNA peroxidase antibody reacts with the DNA component of the nucleosomes. After removal of unbound antibodies by a washing step, the amount of nucleosomes is quantified by the peroxidase retained in the immunocomplex. Peroxidase is determined photometrically with ABTS7 (2,2′-Azido-[3-ethylbenzthiazolin-sulfonate]) as substrate.

For example, Mm5MT breast cancer cells may be plated in a 60 mm dishes at a density of 1×106 cells per dish. Cells are then treated with test compound, and allowed to incubate for 16-24 hours at 37° C. After the incubation, cells are collected, centrifuged, and the supernatant is removed. The cell pellet in each tube is then re-suspended in lysis buffer for 30 minutes. The lysates are then centrifuged and aliquots of the supernatant (i.e., the cytoplasmic fraction) are transferred into a streptavidin-coated dish. Care is taken not to shake the lysed pellets (i.e. cell nuclei containing high molecular weight, unfragmented DNA) in the dish. Samples may then be analyzed.

Alternatively, the cells can be treated with ML-7 for 16-24 hours. The floating cells and the attached cells (released by trypsinization) are then collected and stained with Annexin V: the cells are incubated with 5 μl of FITC-conjugated Annexin V (Pharmingen, San Diego, Calif.) and 10 μl of propidium iodide (50 pg/ml) for 15 min in the dark at 25° C. After incubation, 400 μl of binding buffer is added per sample and cells are analyzed cytofluorimetrically using a Coulter Epics Elite ESP flow cytometer (Ex: 488 nm, Em: 585 nm). At least 10,000 cells are counted per analysis and cells that stain positive for Annexin V and PI are considered apoptotic.

Fold stimulation (FS=ODₘₐₓ/ODᵥₑₕ), an indicator of apoptotic response, is determined for each compound tested at a given concentration. EC₅₀ values may also be determined by evaluating a series of concentrations of the test compound.

Statistically significant increases in apoptosis (i.e., greater than 2 fold stimulation at a concentration of about 30 μM are further indicative that the compound is useful for treating neoplastic lesions. Preferably, the EC50 value for apoptotic activity should be less than 30 μM for the compound to be further considered for potential use for treating neoplastic lesions. EC₅₀ is herein defined as the concentration that causes 50% induction of apoptosis relative to vehicle treatment.

This invention is further illustrated by the following examples, which should not be construed as limiting. However, the disclosure set forth herein is intended to encompass any biologic anticancer agent useful against any tumor cell type for which resistance can be developed. The following Examples are intended to further illustrate certain preferred embodiments of the invention and are not limiting in nature. The contents of all references, patents and published applications cited throughout this application are hereby incorporated by reference herein.

### EXAMPLES

### Example 1

### Myosin Light Chain Dephosphorylation and Apoptosis

A. Studies with Actinomycin D

NIH 3T3 fibroblasts (ATCC Accession No. CRL-1658) were treated with actinomycin D, which is a compound known to rapidly induce cell death, to determine its effect on MLC-P.

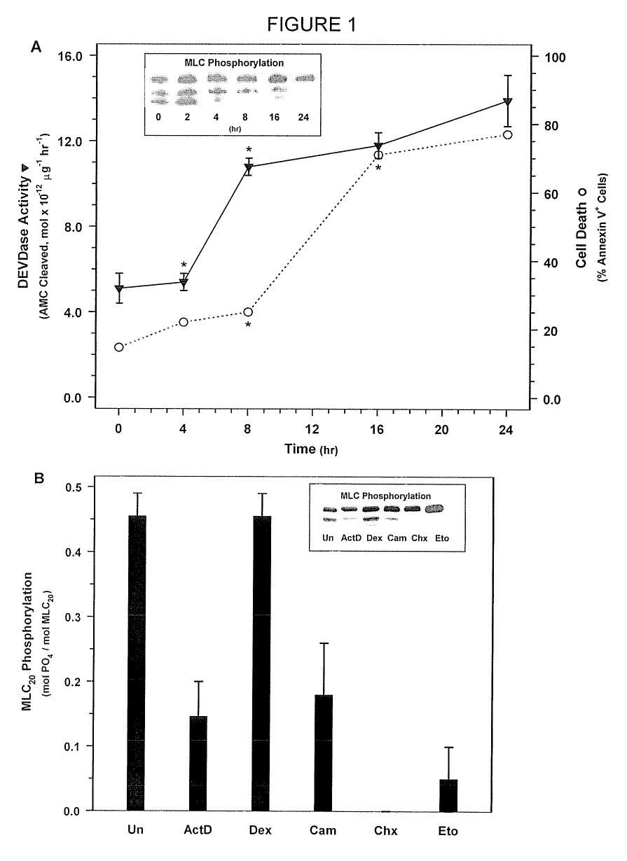

NIH 3T3 fibroblasts were obtained from ATCC and were grown in Dulbecco's Modified Eagle Medium (DMEM) (Gibco BRL, Gaithersburg, Md.) supplemented with 10% FBS and 1% penicillin and streptomycin in a 37° C. incubator. 3T3 cells were treated with 500 nM actinomycin D in DMEM containing 0.5% FBS without antibiotics for the 0, 2, 4, 8, 16, or 24 hours. Cells were then washed with phosphate buffered saline (PBS). Floating cells were collected by centrifugation and combined with attached cells that were harvested by trypsinization. MLC₂₀ phosphorylation was quantified be Western blotting with an anti-MLC₂₀ antibody (FIG. 1). In short, the harvested cells were treated with ice cold 10% TCA, 10 mM DTT to precipitate the cellular proteins. The pellets were washed with acetone, dissolved in 9 M urea, 10 mM DTT, 20 mM Tris, pH 7.5 and separated using glycerol-urea polyacrylamide gel electrophoresis (PAGE). The proteins were transferred to nitrocellulose, the un-, mono-, and di-phosphorylated forms of MLC₂₀ were identified using an affinity purified antibody to MLC₂₀ and horseradish peroxidase-linked secondary antibody (Jackson ImmunoResearch, West Grove, Pa.). Protein bands were visualized with ECL reagent and the stoichiometry of phosphorylation (mol PO₄/mol MLC₂₀) was calculated as described (Obara et al., 1989, “Okadaic acid, a phosphatase inhibitor, produces a Ca2+ and calmodulin-independent contraction of lamb tracheal smooth muscle,” Pfluger's Archiv. 414: 134-138).

The treatment with actinomycin D resulted in an initial increase in MLC₂₀ phosphorylation that was followed by MLC₂₀ dephosphorylation (FIG. 1A, inset; un-, mono-, and di-phosphorylated MLC₂₀ from top to bottom). This initial increase in MLC-P was consistent with previous reports that correlated cell blebbing with increases in MLC-P during the early stages of apoptosis (Mills et al., 1998; and Sebbagh et al., 2001). Following this initial increase, MLC were rapidly dephosphorylated. MLC-DP appeared to precede cell death and MLC-DP was not due to proteolysis of MLC₂₀, myosin II or MLCK because Western blot analyses showed the presence of similar amounts of these proteins at all time points (data not shown). Moreover, MLC-DP preceded caspase activation (FIG. 1A), suggesting that it is a relatively early event in apoptosis.

B. Studies with Agents that Induce Apoptosis

The stoichiometry of MLC₂₀ phosphorylation (FIG. 1B, inset) was quantified using the urea gel/Western blotting method described above in untreated (Un) pig pulmonary artery smooth muscle cells (SMC) and SMC treated with 10 μM dexamethasone (Dex), 10 μM actinomycin D (ActD), 100 μM cycloheximide (Chx), 2 μM camptothecin (Cam), and 100 μM etoposide (Eto) for 24 hrs (mean+/−SE, n=3).

MLC₂₀ were also dephosphorylated when primary cultures of pig pulmonary artery smooth muscle cells were treated with actinomycin D, cycloheximide, camptothecin and etoposide, agents that induce apoptosis (FIG. 1B). Dexamethasone, which induces apoptosis in other cell types (Harvey et al., 1998) neither dephosphorylated MLC₂₀ nor induced smooth muscle cell death. These data suggest a specific link between MLC₂₀ dephosphorylation and cell death.

### Example 2

### MLC-DP Triggers Cell Death

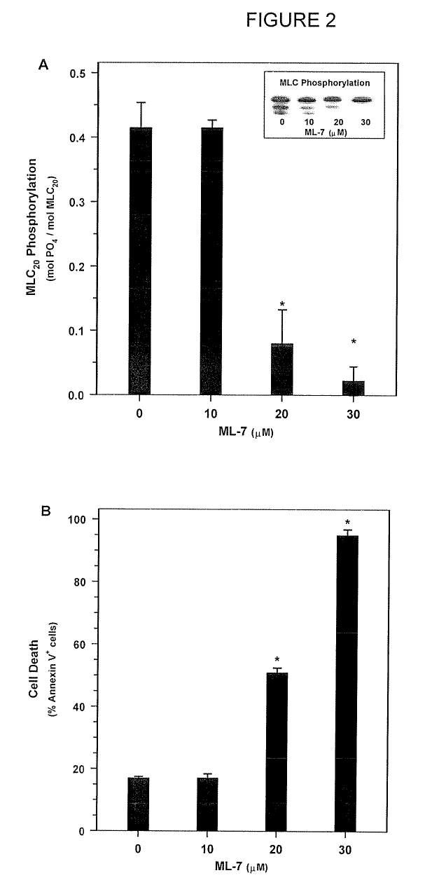

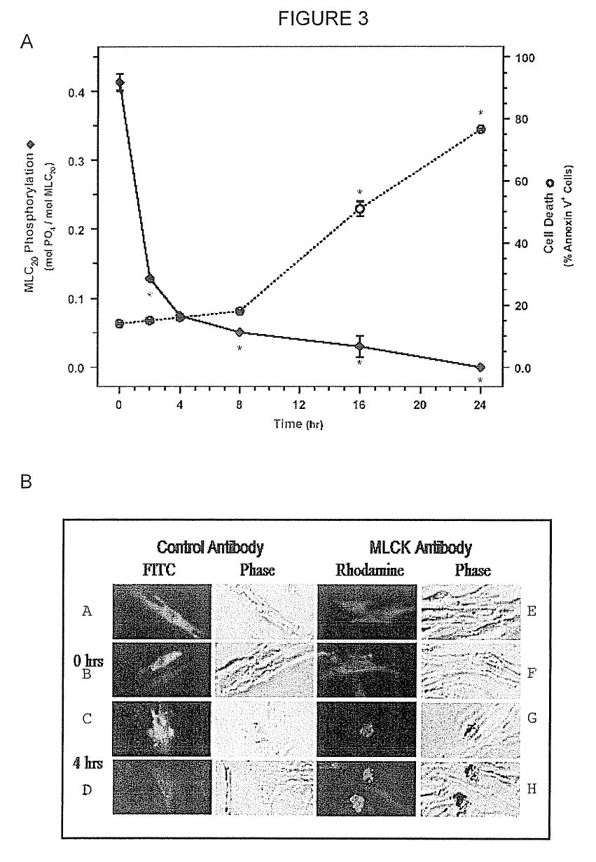

Next, in order to determine whether MLC-DP can trigger cell death, SMC were treated with ML-7 or KT5926, two inhibitors of MLCK (Nakanishi et al., 1990; Garcia, 1998; and Bain et al., 2003). Treatment with increasing doses of ML-7 (0, 10, 20, and 30 μM concentrations) for 16 hours resulted in a dose-dependent decrease in MLC₂₀ phosphorylation (FIG. 2A) and a corresponding increase in cell death (FIG. 2B). Similarly, treatment with KT5926 resulted in cell death, including genome digestion, in NIH 3T3 cells (data not shown). As is the case with non-muscle cells induced with actinomycin D, MLC-DP was an early event in cell death in SMC treated with 20 μM ML-7 (FIG. 3A). Thus, MLC₂₀ dephosphorylation precedes the onset of apoptosis and inhibiting MLCK appears to be sufficient to induce cell death.

### Example 3

### MLCK Inhibitory Antibody Triggers Apoptosis

The role of MLC-DP in apoptosis was investigated further by microinjecting SMC with an affinity purified, inhibitory antibody to MLCK (de Lanerolle, 1981). An affinity purified, goat anti-human IgG was used as a control. Each antibody was mixed separately with either rhodamine-labeled dextran or fluorescein-labeled dextran as an injection marker. Results were mean±SEM for 3 experiments (*p value <0.01 using a paired T test). FIG. 3B shows confocal images of cells microinjected with a control antibody or an affinity-purified inhibitory antibody to MLCK immediately following microinjection (Panels A, B, E, F) and 4 hrs later (C, D, G, H). The control antibody (affinity-purified goat anti-human IgG) was mixed with FITC-labeled dextran prior to injection and the MLCK antibody was mixed with rhodamine-labeled dextran.

Three to four hours after injection, the cells were scored for morphological markers of cell death without prior knowledge of which antibody had been mixed with which marker. While 86% of the cells injected with the MLCK antibody had morphological changes typical of apoptotic cells, only 17% of the cells injected with the control antibody were apoptotic. FIG. 3B shows representative cells that were injected with either the MLCK antibody (Panels G, H) or with the control antibody (Panels C, D). These results establish that inhibiting MLCK, irrespective of the method used, is sufficient to induce apoptosis.

### Example 4

### Destabilizing the Cytoskeleton Leads to MLC₂₀ Dephosphorylation and Apoptosis

Increasing MLC₂₀ phosphorylation stabilizes actin filaments (Trybus, 1996) and increases cytoskeletal stiffness (Hecht et al., 1996; and Cai, 1998). In contrast, cytochalasin D decreases cytoskeletal stiffness (Cai, 1998) and KT5926 leads to the loss of actin filaments (de Lanerolle, unpublished). Moreover, destabilizing actin filaments with cytochalasin D (Brancolini, 1995) or by expressing the actin-severing protein gelsolin (Kothakota, 1997; and Geng, 1998) or by interfering with integrin signaling by preventing substrate attachment (Puthalakath, 2001) also leads to apoptosis.

Because attachment increases MLC-P and stabilizes actin filaments through integrin signaling (Klemke, 1997; and Chrzanowska-Wodnicka and Burridge, 1996), these studies were designed to determine whether the loss of substrate attachment would induce apoptosis by abrogating integrin signaling and decreasing MLC-P and cytoskeletal stiffness.

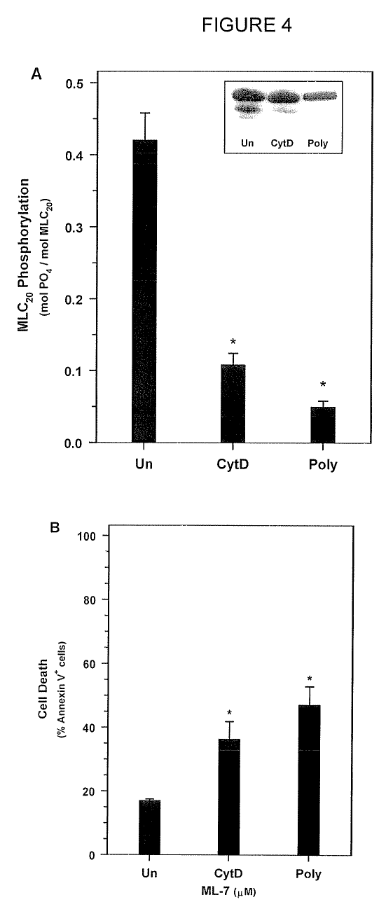

SMC cells were either treated with 10 μM cytochalasin D (CytD) or grown on the non-ionic substrate polyHema to prevent attachment. Treating SMC with 10 μM cytochalasin D (CytD) or growing cells on polyHema (poly) overnight resulted in significant decreases in MLC₂₀ phosphorylation (FIG. 4A) and significant increases in cell death compared to the untreated controls (Un) (FIG. 4B). Results are mean±SEM for 3 experiments (*p value <0.05 using a paired T test).

These results suggest that MLC₂₀ dephosphorylation is a common step that is central to the cell death process induced by cytoskeleton destabilization.

### Example 5

### Caspase Inhibition Protects Against Apoptosis Induced by Inhibiting MLCK

Cytochalasin D and loss of adhesion lead to caspase activation (Puthalakath, 2001; and Frisch and Francis, 1994).

The next set of experiments was designed to investigate whether caspases also are involved in cell death induced by inhibiting MLCK.

Caspases are proteases that are central to the commitment to apoptosis. Caspase activation is generally considered the step that commits cells to apoptosis (Hengartner, 2000, “The biochemistry of apoptosis,” Nature 407: 770-776).

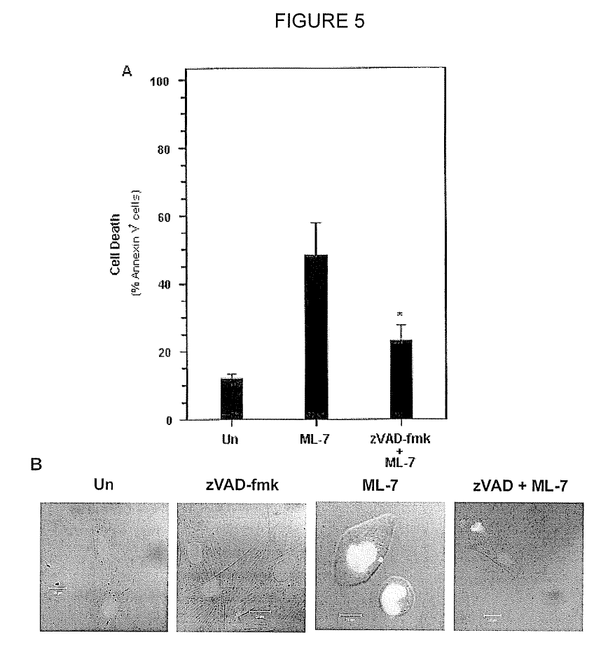

SMC were left untreated, treated with ML-7 alone for 16 hours or treated with 50 μM z-VAD-fmk (caspase) for 1 hr followed by 20 μM ML-7 for 16 hours. FACS analyses showed a significant decrease in the number of apoptotic cells treated with z-VAD-fmk and ML-7 compared to cells treated with ML-7 alone (FIG. 5A). The results represent the mean+/−SE for 3 independent experiments and * indicates a p value <0.05 using a paired T test.

Also, cells were treated with 50 μM z-VAD-fmk for 1 hour, 20 μM ML-7 for 4 hours or 50 μM z-VAD-fmk for 1 hours followed by 20 μM ML-7 for 4 hours (FIG. 5B). The cells were then fixed and stained with TUNEL reagent and DAPI. There were very few TUNEL positive nuclei in untreated cells or cells treated with z-VAD-fmk. Most nuclei in cells treated with ML-7 were TUNEL positive as indicated by the cyan color from the co-localization of the DAPI and FITC-TUNEL stains. z-VAD-fmk decreased the number of TUNEL positive nuclei in ML-7 treated cells.

A. In Vitro Studies in Tumor Cells

The following in vitro studies were performed with either Mm5MT breast cancer cells or MatLyLu prostate cancer cells.

### Example 6

### ML-7 Induces Apoptosis in Mammary and Prostate Cancer Cells

We have demonstrated above that Ml-7 induces apoptosis in smooth muscle cell. To determine if ML-7 has a similar effect on cancer cells, Mm5MT mouse mammary cancer cells and MLL rat prostate cancer cells were treated with varying concentrations of ML-7 for 16 h.

The Mm5MT mouse mammary adenocarcinoma cancer cell line was obtained from American Type Culture Collection (ATCC, Manassas, Va., USA) and was maintained in DMEM medium supplemented with 10% fetal bovine serum (FBS) and 100 U/ml penicillin, 100 μg/ml streptomycin. The MatLy-Lu (MLL) sub-line of the Dunning R-3327 prostate adenocarcinoma (graciously provided by B. Lokeshwar, University of Miami) (Lokeshwar et al., Int. J. Cancer 2002; 98:297-309) was maintained in RPMI1640 medium supplemented with 10% FBS, 250 nM dexamethasone, 100 U/ml penicillin and 100 μg/ml streptomycin.

Mm5MT or MLL cells (200,000 cells per well) were seeded in 6-well dishes one day before drug treatment and cultured as described above. On the day of the experiment, media was changed to DMEM supplemented with 0.5% FBS and the cells were treated with drugs as defined by the specific experimental protocol. ML-7 (Biomol, Plymouth Meeting, Pa.) was incubated with cells for 16 h when used alone. When used in combination with etoposide, ML-7 was added to cells 2 h before adding the indicated concentration of etoposide (Calbiochem, La Jolla, Calif.) and the cells were incubated with ML-7 and etoposide for an additional 16 h. The cells were then treated with trypsin, washed twice with cold PBS and re-suspended in 100 μl of buffer containing 10 mM Hepes, pH 7.4, 140 mM NaCl and 2.5 mM CaCl₂ (binding buffer). Then, 5 μl of FITC-conjugated annexin V (Pharmingen, San Diego, Calif.) and 10 μl of propidium iodide (PI) (50 μg/ml) were added and cells were incubated in the dark at room temperature for 15 min. Next, 400 μl of binding buffer was added per sample and the cells were analyzed cytofluorometrically using a Coulter Epics Elite ESP flow cytometer (Ex: 488 nm, Em: 585 nm). At least 10,000 cells were counted per analysis and cells that stained positive for annexin V and PI were judged to be apoptotic.

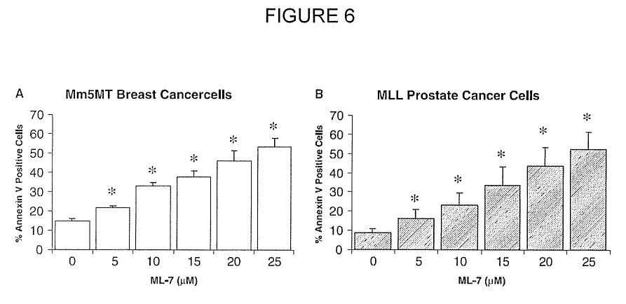

The cells were collected and apoptosis was quantified as described in above. The annexin V and PI positive cells as a percent of total cells, at each concentration of ML-7, are shown (N=4) (*P<0.05 compared to control). FIG. 6 shows that ML-7 induced a dose-dependent increase in apoptotic cells in both Mm5MT and MLL cells.

### Example 7

### ML-7 Potentiates Effects of Other Anti-Cancer Drugs in Prostate Cancer Cells

To determine if MLCK inhibitors could potentiate anti-cancer activities of currently used anti-cancer drugs, such as etoposide, which inhibits topoisomerase (Burden et al., 1996; Sehested and Jensen, 1996), ML-7 (10 μM) was used in combination with varying concentrations of etoposide (0.1 to 1000 μM). The ML-7 was first tested in in vitro to determine if it potentiated induction of apoptosis in prostate cancer cells and breast cancer cells.

Cells were grown in culture dishes in Dulbecco's Modified Eagle Medium (Gibco BRL, Gaithersburg, Md.) supplemented with 10% FBS and 1% penicillin and streptomycin. All drug treatments were performed in DMEM containing 0.5% FBS without antibiotics. Cells were treated with increasing concentrations of etoposide in the presence or absence (control) of either 5 μM (for MatLyLu cells) or 10 μM (for Mm5MT cells) ML-7. Floating cells were collected by centrifugation and attached cells were harvested by trypsin treatment at 16 or 24 hours following the start of treatment. The floating and attached cells were combined and analyzed using conventional methods.

To quantify apoptosis, cells were washed twice with cold PBS, resuspended in 100 μl of 10 mM Hepes, pH 7.4, 140 mM NaCl, 2.5 mM CaCl₂ (Binding Buffer). The cells were then incubated with 5 μl of FITC-conjugated Annexin V (Pharmingen, San Diego, Calif.) and 10 μl of propidium iodide (50 μg/ml) for 15 min in the dark at 25° C. After incubation, 400 μl of binding buffer was added per sample and cells were analyzed cytofluorimetrically using a Coulter Epics Elite ESP flow cytometer (excitation wavelength: 488 nm, emission wavelength: 585 nm). At least 10,000 cells were counted per analysis and cells that stained positive for Annexin V and PI were considered apoptotic.

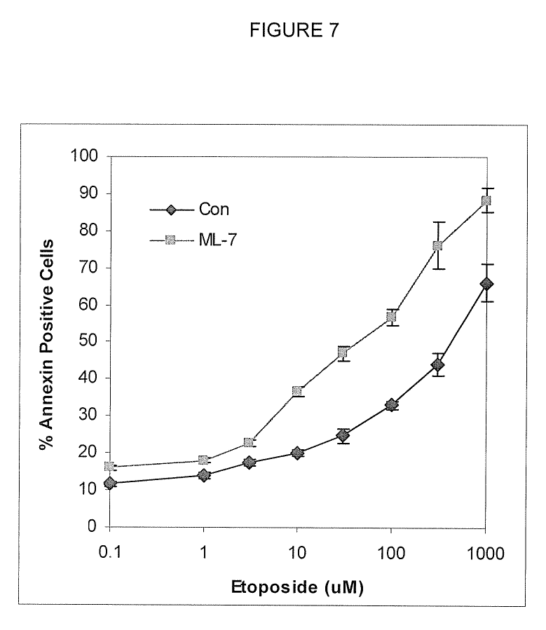

As illustrated in FIG. 7, the half-maximal concentrations for producing apoptosis in MatLyLu cells (prostate cancer cells) was about 410 μM for etoposide alone, and about 40 μM for etoposide plus 5 μM ML-7. This experiment demonstrated that ML-7 potentiates the effects of etoposide.

### Example 8

### ML-7 Potentiates Effects of Other Anti-Cancer Drugs in Breast Cancer Cells

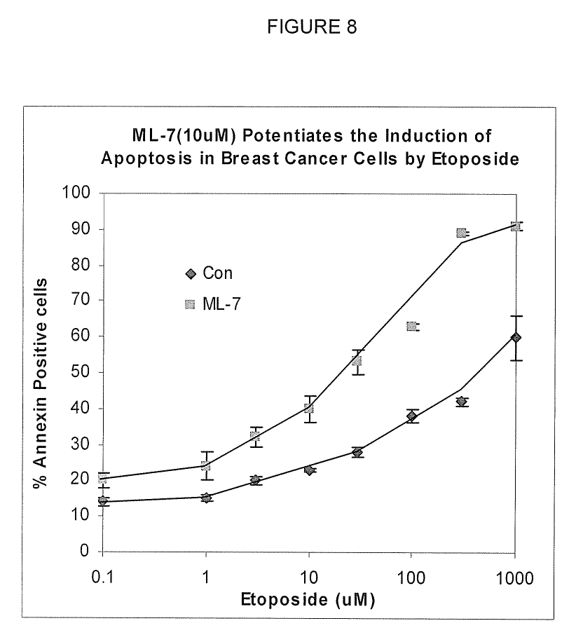

Next, Mm5MT breast cancer cells were treated with ML-7 as described in Example 7. As shown in FIG. 8, the treatment resulted in a shift of the dose-response curve to the left. Extrapolation of the data indicated that the concentration of etoposide required to induce apoptosis in 50% of the Mm5MT cells (breast cancer cells) is about 300 μM for etoposide alone, and about 20 μM plus ML-7. This data indicate that ML-7 increased efficacy of etoposide (induction of apoptosis in breast cancer cells) by over an order of magnitude

Alternatively, TUNEL staining could be performed to study apoptosis. In these assays, cells grown on cover slips are fixed in 4% paraformaldehyde/PBS and permeabilized in a buffer containing 0.1% sodium citrate and 0.1% Triton X-100 in PBS for 2 min on ice. Permeabilized cells are then stained with FITC labeled terminal deoxynucleotidyl transferase dUTP nick-end labeling (TUNEL) enzyme reagent using the In Situ Cell Death Detection Kit (Roche Molecular Biochemicals, Indianapolis, Ind.) according to the manufacturer's instructions. Cover slips are mounted using Vectashield containing DAPI and examined using a Zeiss LSM 510 Laser confocal microscope.

### Example 9

### ML-7 Potentiates Effects of Etoposide in Breast Cancer Cells

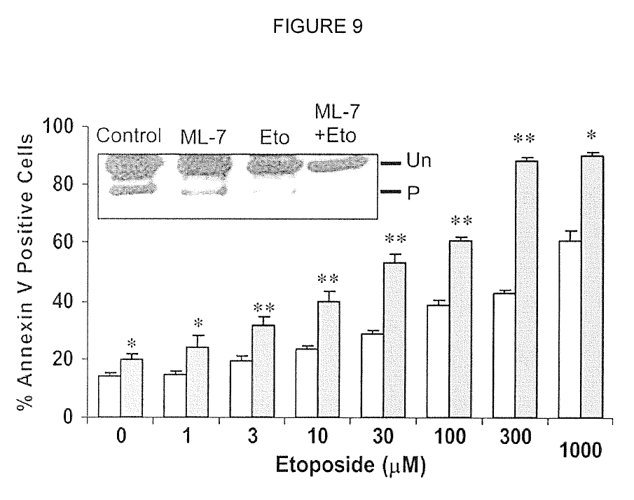

The combined effects of ML-7 and etoposide to induce apoptosis in mouse Mm5MT mammary cancer cells was determined by pre-treating cells with vehicle (FIG. 9, open bar) or 10 μM ML-7 (shaded bar) for 2 h. Varying concentrations of etoposide (1-1000 μM) were then added and apoptosis was quantified 16 h later. Cells were collected 16 h after adding etoposide and apoptosis was quantified by FACS analysis. The annexin V and PI positive cells as a percentage of total cells, at each concentration of etoposide, are shown (N=4, *P<0.05, **P<0.001 vs. etoposide alone). As shown in FIG. 9, ML-7 (10 μM), by itself, significantly increased apoptosis (0 etoposide, FIG. 9) consistent with the data in FIG. 6A, ML-7 also significantly increased the ability of etoposide to induce apoptosis (FIG. 9). A curve-fitting program (Cricket Graph) showed that the concentrations of etoposide required for inducing apoptosis in 50% of the cells was 25.4 μM plus ML-7; and 572 μM minus ML-7.

MLC-P protein was measured by urea/glycerol gel immunoblotting. Mm5MT or MLL cancer cells in 6-well plates were treated with ML-7 (10 or 5 μM, respectively) or etoposide (30 μM) or combination of two at indicated concentrations for 16 h. MLC-P in cancer cells was then quantified by the urea/glycerol gel-immunoblotting method. Briefly, cancer cells were fixed in 10% trichloroacetic acid (TCA) containing 10 mM dithiothreitol (DTT). Cell pellets were washed four times with acetone and protein was extracted by dissolving in buffer containing 9 M urea, 10 mM DTT and 20 mM Tris, pH 8.0. The unphosphorylated and phosphorylated forms of MLC₂₀ were separated using urea/glycerol PAGE, transferred to nitrocellulose and probed with an affinity purified antibody to MLC₂₀. This antibody recognizes the unphosphorylated and phosphorylated forms of MLC₂₀. Immunoreactive bands were visualized using enhanced chemiluminescence (ECL) detection reagents (Amersham Pharmacia Biotech, Piscataway, N.J.).

As shown in the inset of FIG. 9, Mm5MT cells were treated with vehicle (control), 10 μM ML-7, 30 μM etoposide or 10 μM ML-7 and 30 μM etoposide. MLC-P was measured by urea/glycerol gel-immunoblotting as described above. Un and P identify unphosphorylated and phosphorylated MLC₂₀, respectively. Note the decrease in the phosphorylated band in the treated groups compared to the control. This experiment was repeated four times and the data from a representative experiment is shown. The result shows that 10 μM ML-7 and 30 μM etoposide decreased MLC-P in Mm5MT cells and that the combination of the two drugs almost completely eliminated MLC-P (see FIG. 9, inset).

B. Studies Performed in an In Vitro Mammary Cancer Model

### Example 10

### ML-7 has a Chemopreventive Effect in an In Vitro Mammary Cancer Model

Mammary glands obtained from young Balb/c mice that are exposed to 7,12-dimethylbenz(a)anthracene (DMBA) for 24 h in culture to form precancerous lesions in 24 days. The procedure has been successfully used to determine efficacy of chemopreventive agents and is described in detail elsewhere. (Mehta et al. PPARγ ligand and retinoids prevent preneoplastic mammary lesions. J. Natl Cancer Instit 2000; 92:418-23). Briefly, 70 mammary glands from 35 Balb/c mice were divided into seven groups of 10 glands each and incubated in serum-free medium containing insulin, prolactin (5 μg/ml each), aldosterone and hydrocortisone (1 μg/ml each) for 10 days. DMBA (2 μg/ml) was included in the medium for 24 h on day 3. The glands were incubated for an additional 14 days in the absence of hormones except insulin. This allows the regression of the normal mammary alveolar structures whereas the precancerous mammary alveolar lesions (MAL) acquire altered hormonal responsive-ness and do not regress under these conditions. Chemopreventive agents were included in the medium during the first 10 days. The glands were fixed in formalin and stained with alum carmine and evaluated for MAL. Percent inhibition was calculated by comparing the incidence in the control glands with the treated groups.

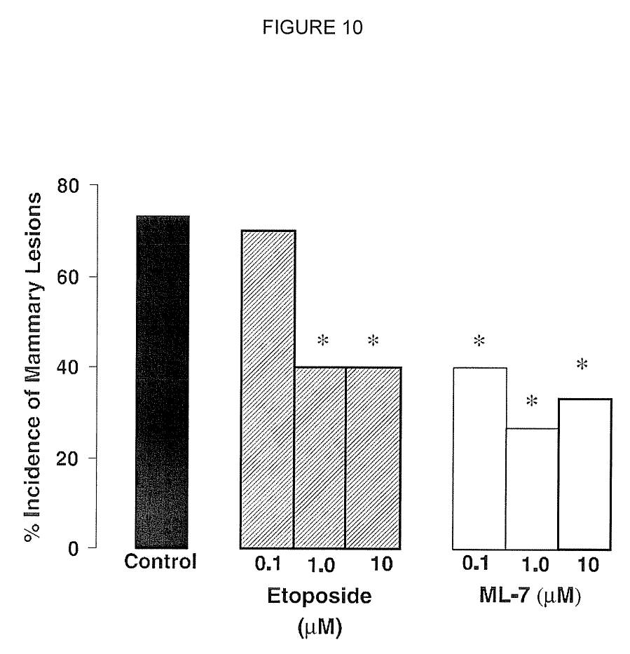

Mammary glands obtained from young Balb/c mice were exposed to DMBA as described above and MAL formation was monitored. The inhibition of MAL formation has previously been successfully used as a parameter to judge the possible efficacy of chemopreventive agents. (Mehta et al. PPARγ ligand and retinoids prevent preneoplastic mammary lesions. J. Natl Cancer Instit 2000; 92:418-23) In the present study, we evaluated effects of etoposide and ML-7 at 0.1, 1.0 and 10.0 μM concentrations on the development of MAL in organ culture. As shown in FIG. 10, etoposide inhibited the incidence of lesion formation at 1 and 10 μM concentration by 40-46% compared to control. Etoposide at 0.1 μM, however, did not affect MAL formation. ML-7 suppressed the development of MAL by 40% even at 0.1 μM and further reduced it to 58% of control at 1 μM. Percentage inhibition was calculated by comparing the incidence in the control glands with the treated groups. Results were subjected to χ² analysis (*P<0.05 compared to control). The differences observed between 0.1 and 10 μM ML-7 were not statistically different. However the inhibition of 58% at 1 μM compared to a 70% incidence in the control glands ( 7/10 glands positive) was significant.

C. In Vivo Studies in Animal Tumor Model

### Example 11

### The Animal Model

Tumor free female MMTV+C3H/HeN mice were obtained from Harlan Sprague Dawley, Inc. (Indianapolis, Ind.), an animal supplier.

K1735 murine melanoma cells or Mm5MT (MMTV-induced) murine mammary carcinoma cells were grown in Dulbecco's Modified Eagle Medium (DMEM) supplemented with 10% fetal calf serum (FCS) and Penn/Strep antibiotic. The cells were maintained in 5% CO₂. The cells were harvested, washed in phosphate buffered saline (PBS), counted and re-suspended in PBS according to the standard protocol (Paul, 1970, CELL AND TISSUE CULTURE, 5th Edition, Churchill Livingstone, Edinburgh, London and New York).

Tumor free mice (15 per group) were anesthetized with ether and injected subcutaneously in the flank with 1×10⁶ K1735 murine melanoma cells or Mm5MT carcinoma cells. One hundred microliter osmotic pumps (Alzet, Palo Alto, Calif.) containing dimethyl sulfoxide (DMSO) (controls) or 27 mM ML-7 in DMSO were implanted in the shoulder during the same surgery. The osmotic pumps were replaced with new osmotic pumps two weeks later.

Tumor progression was monitored by physical examination every other day.

Four weeks after the initial pump was implanted, the mice in the control and ML-7 treated groups were sacrificed and the tumors were removed for histological evaluation.

Alternatively, only half of the mice may be sacrificed at 4 weeks and the other half is not sacrificed until tumors reach a size that requires euthanasia. The objective of keeping the animals longer than the 4 week treatment period would be to determine whether ML-7 has an affect on the survival of the animals.

The effectiveness of the ML-7 therapy was evaluated by physical and histological examinations. When the mice were sacrificed, the tumors were removed, weighed, their dimensions measured, cut into 5 mm cubes, and fixed in formalin for histological evaluation.

TUNEL staining was also performed on formalin fixed sections as previously described to establish that the cells were dying by apoptosis. In brief, the tumor sections were deparaffinized and rehydrated according to standard protocol. Tissue sections were permeabilized by placing slides in 10 mM citrate buffer (pH 6.0) and applying 350 W microwave irradiation for 5 min. Tissue sections were then stained with TMR Red-labelled terminal deoxynucleotidyl transferase dUTP nick-end labeling (TUNEL) enzyme reagent using the In Situ Cell Death Detection kit (Roche Molecular Biochemicals, Indianapolis, Ind.) as described by the manufacturer. Sections stained with the labelling solution without the terminal transferase was used as negative control. Tissue sections were finally mounted using Vectashield containing DAPI and examined using a Zeiss LSM 510 laser confocal microscope.

Tissue sections (0.5 μm thick) were stained with FITC labeled terminal deoxynucleotidyl transferase dUTP nick-end labeling (TUNEL) enzyme reagent using the In Situ Cell Death Detection Kit (Roche Molecular Biochemicals, Indianapolis, Ind.) according to the manufacturer's instructions. Cover slips and tissue sections were mounted using Vectashield containing DAPI. These tissue sections were then examined using a Zeiss LSM 510 Laser confocal microscope.

### Example 12

### Treatment with ML-7 Decreases Tumor Growth in the Mouse Model

Studies with Breast Cancer Cells

Only adult females are used because the cells are estrogen-sensitive.

To produce tumors, Mm5MT murine mammary tumor cells grown in culture were harvested and washed to remove any serum. The cells were suspended in 0.2 mL of PBS and 1,000,000 cells per animal were injected subcutaneously into right flank of syngeneic C3H/HeN mice (anesthetized with ether and whose backs and flanks were shaved and prepped with Betadine).

To deliver ML-7, a small incision was made in the interscapular area at the same time as the cells are implanted. A small subcutaneous pocket was created into which an Alzet osmotic pump (100 microliters) is implanted. These pumps, with release rates of 0.25 microliters/hour, were filled with sterile 50% DMSO in PBS or 27 mM ML-7 dissolved in 50% DMSO in PBS. The wounds were then closed with surgical staples. After each procedure, animals' recovery from anesthesia was closely observed. All procedures were performed under sterile conditions as approved by the UIC Animal Care Committee.

Initially, the researchers were concerned about giving ML-7 to mice because ML-7 was thought to cause reduction in blood pressure or cause gastrointestinal problems, as ML-7 relaxes smooth muscles. However, the mice tolerated these doses of ML-7 without any obvious discomfort. Mice and rats implanted with pumps primed with 27 mM ML-7 were active, ate well, put on weight and visually seemed to be unaffected by the ML-7. Moreover, blood pressure, heart rate and respiratory parameters did not change dramatically in rats that received bolus injections of ML-7 that were designed to produce a plasma level of 20 μM (N. Zigon, Belgrade University, personal communication).

The mice were observed daily following the surgery for any signs of bleeding, infection, wound dehiscence, overlying skin necrosis, lethargy, respiratory distress or inability to get to food and water. Tumor growth was also monitored every other day. After two weeks the old pumps were removed and a new ones implanted as previously described, because these small Alzet pumps only deliver drug for 14 days. The same subcutaneous pocket created during the first procedure was used for the replacement pump. Four weeks after tumor implantation, the mice in this study were sacrificed with ether and the tumors were removed, weighed and processed for histology.

Sections were prepared from tumors removed from mice receiving DMSO or ML-7 and stained with TUNEL reagent (to identify apoptotic cells) and DAPI (to visualize nuclei).

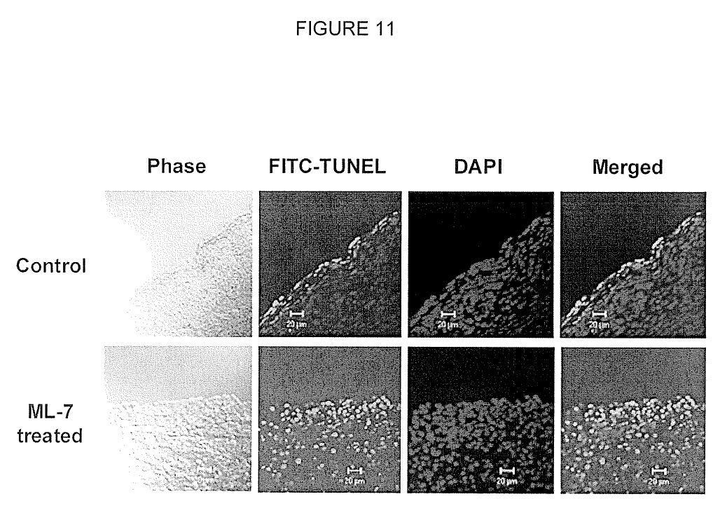

Photomicrographs demonstrated the presence of more apoptotic cells in the sections from the mice receiving ML-7 than in the controls (FIG. 11). Quantification of the TUNEL-positive nuclei in 500 cells from randomly chosen sections in each group showed that 2.5% and 15.6% of the nuclei were TUNEL positive in tumors removed from control mice and mice receiving ML-7, respectively. Quantification of the weights of the tumors revealed the following results (Table D).

TABLE D

| | No. of | |
|---|---|---|
| Treatment Group | animals | Tumor weight |
| DMSO Controls | N = 13 | 1.23 +/− 0.30 gm (mean +/− SE) |
| 27 mM ML-7 | N = 14 | 0.56 +/− 0.20 gm (mean +/− SE) |

These studies suggested that ML-7 was well tolerated well by the mice. More importantly, these studies showed that treatment with ML-7 induced apoptosis in the tumor cells and retarded breast cancer cell growth in mice.

Studies on Melanoma Cells

Two experiments were performed using K1735 melanoma cells for injection into experimental animals, as described above using breast cancer cells. Sections were prepared from tumors removed from mice receiving DMSO or ML-7 and stained with TUNEL reagent (to identify apoptotic cells) and DAPI (to visualize nuclei).

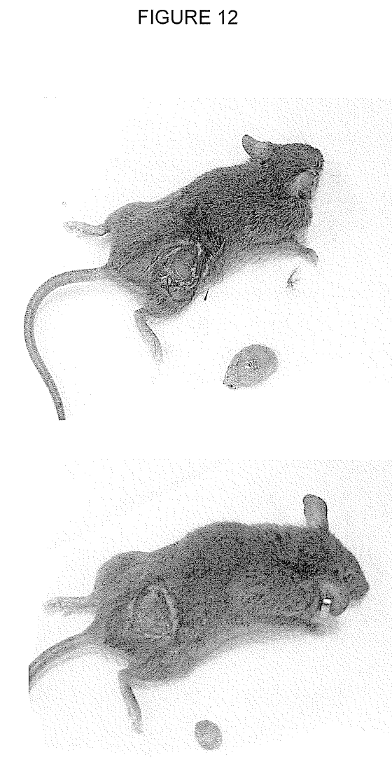

As shown in FIG. 12, a control mouse receiving 50% DMSO in PBS developed a tumor weighing 0.9 grams with dimensions of ˜1.6 cm×1.1 cm×1 cm. In contrast, a mouse receiving 27 mM ML-7 had a much smaller tumor that weighed 0.17 grams and measured ˜0.5 cm×0.4 cm×0.4 cm (FIG. 12).

These experiments indicate that ML-7 is also capable of inhibiting the growth of melanoma cells in mice.

### Example 13

### ML-7 has an Additive Tumoricidal Effect with Etoposide on Mammary Cancer in Mice

To investigate the anticancer activity of ML-7 in vivo, Mm5MT cells grown in culture were harvested immediately before injection into syngeneic MMTV-C3H/HeN mice. Cells were washed to remove serum and 10⁶ cells were resuspended in 100 μl of serum-free DMEM. Healthy, MMTV-free female mice (14-20 weeks old) were anesthetized with ether and 10⁶ cells were injected subcutaneously into the right flank. The mice were randomly divided into four groups of five mice each, and treated with vehicle, ML-7, etoposide or ML-7 plus etoposide. Drug administration was started 1 week after the cells were injected when the mice had developed palpable tumors. To deliver ML-7, a small horizontal incision was made in the interscapular area and a 200 μl osmotic pump (Alzet, Cupertino, Calif.) filled with either 27 mM ML-7 in 50% DMSO or 50% DMSO (vehicle control) was implanted and the wound closed. When given, 25 mg/kg etoposide was injected intraperitoneally on the first 3 days of every week for 4 weeks (days 7-9, 14-16, 21-23 and 28-30). The mice were sacrificed with ether after 4 weeks of drug administration and tumors were removed, weighed and processed for analysis.

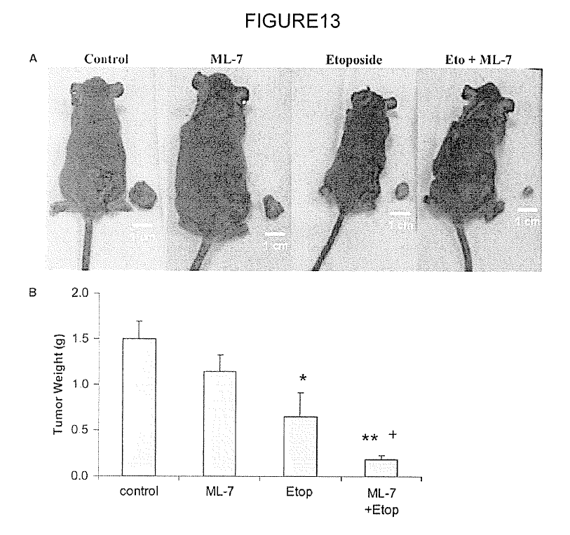

Physical examination of the mice showed that mice treated with vehicle, ML-7 or etoposide alone tolerated these drugs without visible signs of discomfort generally associated with systemic relaxation of smooth muscles. ML-7 and etoposide both decreased tumor growth, but only the etoposide effect was statistically significant (P<0.05) compared to mice receiving vehicle. The combination of ML-7 and etoposide dramatically reduced tumor growth compared to mice receiving vehicle (88.5% inhibition of tumor growth, P<0.001) and to mice receiving etoposide alone (P<0.05) (FIG. 13).

Excised tumors were gently patted dry and weighed using a Mettler digital balance. Tumors were sectioned into 2 mm slices, fixed in 10% neutral buffered formalin, routinely processed and embedded in paraffin. Five micron sections demonstrating the entire surface were stained by hematoxylin and eosin and examined by a surgical pathologist blinded to the experimental conditions. Photomicrographs documenting the entire section were collected and areas of necrotic and viable tumor were determined using the manual tracing tools within MetaMorph 6.2 (Universal Imaging Corporation, Downingtown, Pa.).

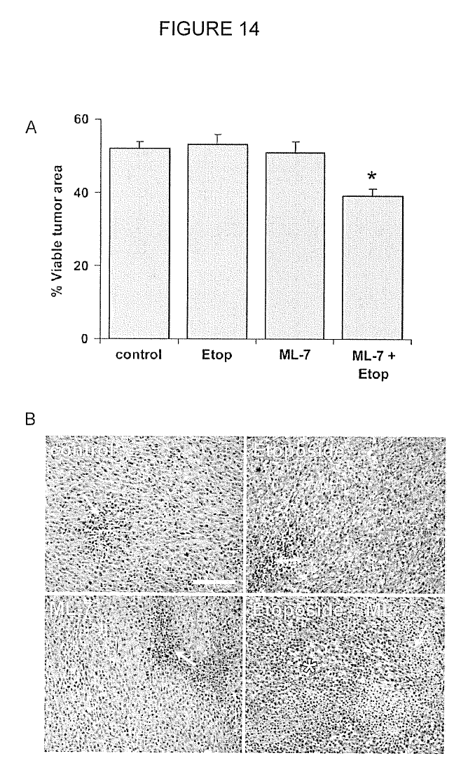

Histological analysis revealed significant necrosis within control, ML-7-treated, etoposide-treated, and ML-7 plus etoposide-treated mice. Blinded examination of photomicrographs and quantification of areas of necrotic and viable tumor using manual tracing tools within MetaMorph 6.2 revealed significantly less viable tumor area in mice treated with etoposide and ML-7 compared to control (N=5, *P<0.05) (FIG. 14A). It was apparent on further examination that the distribution of necrosis in control, etoposide treated and ML-7-treated mice was predominantly confined to the center of the tumor, a pattern that is typically seen in rapidly growing tumors. Away from these central areas, small foci of apoptosis were apparent but adjacent tumor was viable, with intact cell adhesions, as evident by tumor cell cohesion, and readily identifiable mitotic figures. Representative medium power images are shown of tumors from control, etoposide-treated, ML-7 treated, and etoposide plus ML-7-treated mice (FIG. 14B). Limited areas of necrosis are present in tumors from etoposide and ML-7 treated mice (arrow), but adjacent tumor is viable and mitotic figures are easily found. In contrast, larger areas of necrosis are present in tumors from etoposide plus ML-7 treated mice (arrow) and adjacent viable tumor shows signs of impending apoptosis, including incohesion (asterisk), where the necrosis was distributed in a predominantly perivascular pattern (FIG. 14B, Bar=100 μm). This pattern was clearly distinguishable from that seen in the other tumors and suggested that necrosis may have been induced by blood-borne agents, (i.e., ML-7 and etoposide). Individual cells within these areas of necrosis were characterized by dense eosinophilic cytoplasm and shrunken fragmented nuclei, a morphology typical of apoptosis. Cells adjacent to these areas, that were not frankly necrotic, generally showed early signs of cell death, including discohesion, vacuolization and absence of mitoses. Thus, the observed in vivo synergy between ML-7 and etoposide caused a pattern of tumor necrosis consistent with the enhanced apoptosis observed in vitro.

### Example 15

### ML-7 Induces Apoptosis in Prostate Cancer Cells and has Tumoricidal Effects on Rat Prostate Cancer

To determine whether ML-7 stimulated the ability of etoposide to induce apoptosis and retard tumor growth more generally in tumor cells, the effects of ML-7 and etoposide on MLL prostate cancer cells were examined. MLL cells grown in culture were harvested and washed in serum-free Hank's buffer. The cells were suspended in 500 μl serum-free Hank's media and 10⁶ cells were injected subcutaneously into the right flank of 12-week old male Copenhagen rats anesthetized with ether. The cells were allowed to grow and drug treatment was started 5 days after inoculation when the rats had developed palpable tumors. Rats were randomly divided into four groups, five in each group, and received injections of ML-7 or vehicle via the jugular vein every 4 days for 2 weeks. ML-7 was used at the dose of 35 mg/kg. In mice receiving etoposide the drug was injected IP at the maximum tolerant dose of 50 mg/m² daily. Rats were sacrificed with ether 14 days after the start of drug treatment, and tumors removed, weighed and processed as described below.

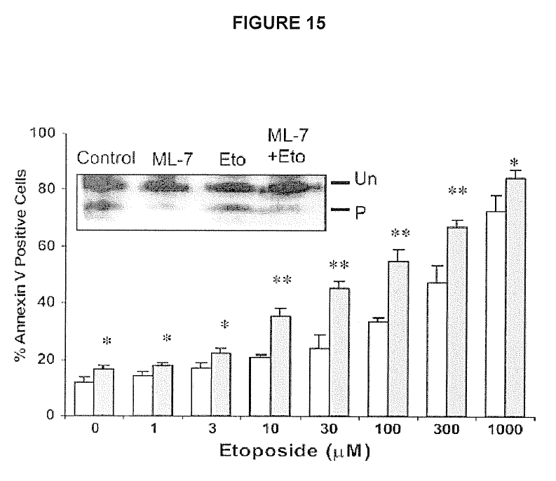

In these experiments, MLL cells grown in culture were pre-treated with 5 μM ML-7 before adding varying concentrations of etoposide from 1 to 1000 μM. ML-7 significantly increased the apoptotic effect of etoposide when compared with cells treated with etoposide alone, and decreased the concentration required for inducing apoptosis in 50% of the cells from 376 μM (no ML-7) to 68 μM (with ML-7, FIG. 15). Urea/glycerol gel-immunoblotting showed that 5 μM ML-7 decreased MLC-P and that 30 μM etoposide resulted in a smaller decrease in MLC-P in MLL cells. When used together, MLC-P was decreased to a level comparable to ML-7 alone (FIG. 15, inset). For experimentation, MLL cells were pretreated with vehicle (open bars) or 5 μM ML-7 (stippled bars) for 2 h prior to adding the indicated concentrations of etoposide. Cells were collected 16 h after adding etoposide and apoptosis was quantified by FACS analysis. The annexin V and PI positive cells as a percentage of total cells, at each concentration of etoposide, are shown (N=4, *P<0.05 and **P<0.01 vs. etoposide alone). See FIG. 15 (Inset). MLL cells were treated with vehicle (control), 5 μM ML-7, 30 μM etoposide or 5 μM ML-7 and 30 μM etoposide. MLC-P was measured by urea/glycerol gel-immunoblotting as described above. Un and P identify unphosphorylated and phosphorylated MLC₂₀, respectively. ML-7 alone and with 30 μM etoposide, resulted in a substantial decrease in the phosphorylated MLC₂₀ band where as etoposide resulted in a smaller decrease in MLC-P. This experiment was repeated four times and the data from a representative experiment is shown.

### Example 16

### ML-7 and Etoposide have a Potent, Additive Tumoricidal Effect on Prostate Tumors

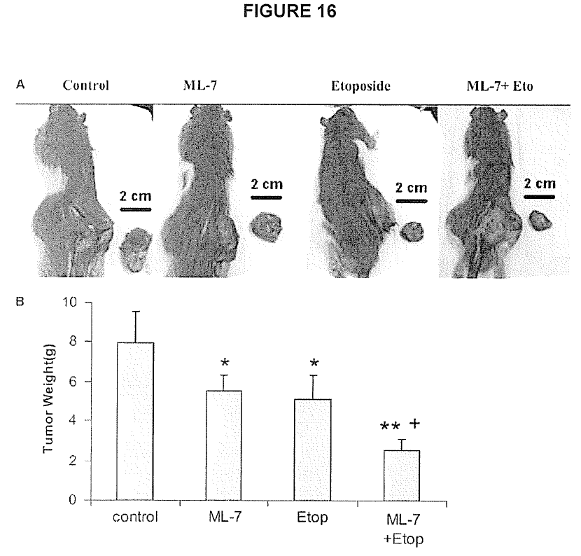

To test the anticancer effect of ML-7 in a rat prostate cancer model, ML-7 or vehicle was administered by intrajugular injection, and etoposide administered by intraperitoneal injection. Male Copenhagen rats were inoculated with MLL cells as described above. Drug treatment was started 5 days later when the rats had developed palpable tumors. The rats were sacrificed after 14 days of drug treatment and tumor weight and body weight were recorded. Tumors were removed from representative rats in each treatment and compared with control (see FIG. 16 A) and the means±SE for tumor weight in each group is calculated (N=4, *P<0.05, **P<0.0001 vs. vehicle control, +P<0.05 vs. etoposide alone) (see FIG. 16B). As in experiments performed as described above using mice, rats appeared to tolerate the individual drugs or vehicle without obvious discomfort. Rats receiving both ML-7 and etoposide, however, appeared to be more lethargic and lost on average 15% of their initial body weight. ML-7 or etoposide alone significantly inhibited prostate tumor growth and decreased tumor weight by 29.6% and 43.3%, respectively (P<0.05 vs. vehicle control). The combination ML-7 and etoposide further retarded tumor growth and decreased tumor weight by 79.1% compared to the vehicle control (P<0.001 vs. vehicle control) (FIG. 16).

Furthermore, TUNEL staining showed more apoptotic cells in sections from rats receiving ML-7 or etoposide compared to vehicle control.

Tumor sections were deparaffinized and rehydrated according to standard procedures. Tissue sections were permeabilized by placing slides in 10 mM citrate buffer (pH 6.0) and applying 350 W microwave irradiation for 5 minutes. Tissue sections were then stained with TMR Red-labeled terminal deoxynucleotidyl transferase dUTP nick-end labeling (TUNEL) enzyme reagent using the In Situ Cell Death Detection kit (Roche Molecular Biochemicals, Indianapolis, Ind.) as described by the manufacturer. Sections stained with labeling solution without the terminal transferase was used as negative control. Tissue sections were finally mounted using Vectashield containing DAPI and examined using a Zeiss LSM 510 laser confocal microscope.

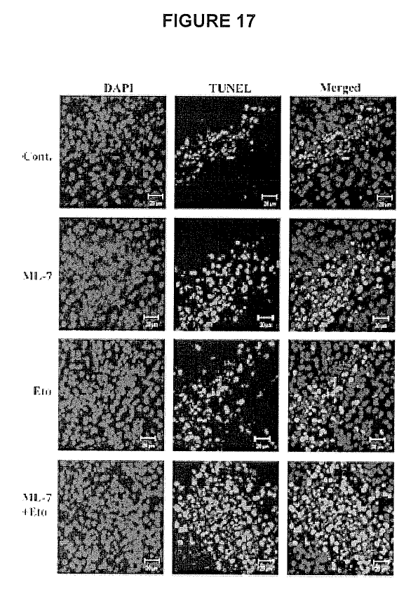

Importantly, the combination of ML-7 and etoposide further increased the number of apoptotic cells detected. Quantification of TUNEL-positive nuclei in 300 cells from randomly chosen fields in each group showed that 19.2%, 40.6%, 35.8% and 66.7% of the nuclei were TUNEL positive in control, ML-7-treated, etoposide-treated, and ML-7 plus etoposide-treated tumors, respectively (FIG. 17, Bar=20 μm).

It is therefore intended that the foregoing detailed description be regarded as illustrative rather than limiting, and that it be understood that it is the following claims, including all equivalents, that are intended to define the spirit and scope of this invention.

### REFERENCES

JP 62-1 64,626
U.S. Pat. No. 5,015,578
U.S. Pat. No. 4,935,415
U.S. Pat. No. 5,427,916
JP 01-149,791
JP 01-246,288
JP 02-258,724
JP 03-72,485
JP 01-143,877
JP 02-09,819
JP 03-220,194
WO 891071 05
WO 91 109034
EP 41 0,389
EP 296,110
JP 63-295,988
JP 62-240,689
JP 61-268,687
JP 62-1 55,284
JP 62-1 55,285
JP 62-1 20,388
JP 63-295,589
WO 88107045
EP 323,171.
Abeloff M D, et al., Alopecia and Cutaneous Complications, p. 755-56
Abeloff M D, Armitage J O, Lichter A S, and Niederhuber J E, (eds), Clinical Oncology, Churchill Livingston, N.Y., 1992.
Amano, M., Ito, M., Kimura, K., Fukata, Y., Chihara, K., Kakano, T., Matsuura, Y., and Kaibuchi, K. Phosphorylation and activation of myosin by rho-associated kinase (rho) kinase. J. Biol. Chem., 271:20, 246-249, 1996.
Bain, J., McLauchlan, H., Elliott, M., and Cohen, P. (2003) The specificities of protein kinase inhibitors: an update. Biochem J. 371: 199-204, 2003.
Bergulund, J. J., Riegler, M., Zolotarevsky, Y., Wenzl, E. and Turner, J. R. Regulation of human jejunal transmucosal resistance and MLC phosphorylation by Na+-glucose cotransport. Am. J. Physiol., 281: G1487-G1493, 2001.
Brancolini, C., Benedetti, M., and Schneider, C. Microfilament reorganization during apoptosis: the role of Gas2, a possible substrate for ICE-like proteases. EMBO J. 14: 51 79-5190, 1995.
Burden, D. A., Kingma, P. S., Froelich-Ammon, S. J., Bjornsti, M-A., Patchan, M. W., Thompson, R. B. and Osheroff, N. Topoisomerase II-etoposide interactions direct the formation of drug-induced enzyme-DNA cleavage complexes. J. Biol. Chem. 271: 29238-29244, 1996.
Cai, S., Pestic-Dragovich, L., O'Donnell, M. E., Wang, N., Ingber, D., Elson, E. and de Lanerolle, P. Regulation of cytoskeletal dynamics and cell growth by myosin light chain phosphorylation. Am. J. Physiol., 275: C1349-C1356, 1998.
Chen, L., Xin, X., Eckhart, A. D., Yang, N., and Faber, J. E. Regulation of vascular smooth muscle growth by alpha 1-adrenoreceptor subtypes in vitro and in situ. J. Biol. Chem. 270: 30980-30988, 1995.
Chew, T. L., Masaracchia, R. A., Goeckeler, Z. M., and Wysolmerski, R. B. Phosphorylation of non-muscle myosin II regulatory light chain by p21-activated kinase (gamma-PAK). J. Muscle Res. Cell Motil. 19: 839-854, 1998.
Choi, O. H., Adelstein, R. S., and Beaven, M. A. Secretion from rat basophilic RBL-2H3 cells is associated with diphosphorylation of myosin light chains by myosin light chain kinase as well as phosphorylation by PKC. J. Biol. Chem., 269: 536-541, 1994.
Chrzanowska-Wodnicka, M. and Burridge, K. Rho-stimulated contractility drives formation of stress fibers and focal adhesions. J. Cell Biol., 133: 1403-1415, 1996.
de Lanerolle, P. and Paul, R. J. Myosin phosphorylation/dephosphorylation and regulation of airway smooth muscle contractility. Am. J. Physiol., 261: L1-L14, 1991.
de Lanerolle, P., Adelstein, R. S., Feramisco, J. R. and Burridge, K. Characterization of antibodies to smooth muscle myosin kinase and their use in localizing myosin kinase in nonmuscle cells. Proc. Natl. Acad. Sci. USA, 78: 4738-4742, 1981.
De Lozanne, A. and Spudich, J. A. Disruption of the Dictyostelium myosin heavy chain gene by homologous recombination. Science, 236: 1086-1091, 1987.
Duke & Cohen, “Morphological And Biochemical Assays Of Apoptosis,” Current Protocols In Immunology, Coligan et al., eds., 3.1 7.1-3.17.16, 1992.
Ettiene-Manneville, S., and Hall, A. Rho GTPases in cell biology. Nature, 4201629-635, 2002.
Feleszko, W., Mlynarczuk, I., Olszewska, D., Jalili, A., Grzela, T., Lasek, W., Hoser, G., Korczak-Kowalska, G. and Jakobisiak, M, Lovastatin potentiates antitumor activity of doxorubicin in murine melanoma via an apoptosis-dependent mechanism. Int J Cancer. 100: 1 1 1 1-1 18, 2002.
Fishkind, D. J., Cao, L-G. and Wang, Y-L. Microinjection of the catalytic fragment of myosin light chain kinase into dividing cells: Effects on mitosis and cytokinesis. J. Cell Biol., 1 14: 967-975, 1991.
Fogh, J., and Trempe, G. In: Human Tumor Cells in Vitro, J. Fogh (eds.), Plenum Press, New York, pp. 1 15-159, 1975.
Friesen, C., Fulda, S. and Debatin, K. M. Cytotoxic drugs and the CD95 pathway. Leukemia 13: 1854-1858, 1999.
Frisch, S. M. and Francis, H. Disruption of epithelial cell-matrix interactions induces apoptosis. J. Cell Biol., 124: 61 9-626, 1994.
Gandhi, S., Lorimer, D. D. and de Lanerolle, P. Expression of a mutant myosin light chain that cannot be phosphorylated increases paracellular permeability. Am. J. Physiol., 272: F214-F221, 1997.
Garcia, J. G., Verin, A. D., Herenyiova, M., and English, D. Adherent neutrophils activate endothelial myosin light chain kinase: role in transendothelial migration. J. Appl. Physiol. 84: 181 7-1821, 1998;
Geng, Y. J., Azuma, T., Tang, J. X., Hartwig, J. H., Muszynski, M., Wu, Q., Libby, P. and Kwiatkowski, D. J. Caspase-3-induced gelsolin fragmentation contributes to actin cytoskeletal collapse, nucleolysis, and apoptosis of vascular smooth muscle cells exposed to proinflammatory cytokines. Eur. J. Cell Biol., 77: 294-302, 1998.
Harvey, K. J., Blomquist, J. F., and Ucker, D. S. Commitment and effector phases of the physiological cell death pathway elucidated with respect to Bcl-2, caspase, and cyclin-dependent kinase activities. Mol. Cell. Biol. 18: 291 2-2922, 1998.
Harvey, K. J., Lukovic, D., and Ucker, D. S. Caspase-dependent Cdk activity is a requisite effector of apoptotic death events. J Cell Biol. 148: 59-72, 2000.
Hecht, G., Pestic, L., Nikcevic, G., Koutsouris, A., Tripuraneni, J., Lorimer, D. D., Nowak, G., Guerriero Jr., V., Elson, E. L. and de Lanerolle, P. Expression of the catalytic domain of myosin light chain kinase increases paracellular permeability. Am. J. Physiol., 271: C1678-C1684, 1996.
Hengartner, M. O. (2000). The biochemistry of apoptosis. Nature 407: 770-776.
Herbst, R. S. and Khuri, F. R. Mode of action of docetaxel—a basis for combination with novel anticancer agents. Cancer Treat Rev. 29: 407-41 5, 2003.
Isemura M, et al., “Myosin light chain kinase inhibitors ML-7 and ML-9 inhibit mouse lung carcinoma cell attachment to the fibronectin substratum,” Cell Bio. Int. Rep. 15(10):965-972, 1991.
Jaffer, Z. M. and Chernoff, J. p21-activated kinases: three more join the Pak. Int. J. Biochem. Cell Biol., 34: 71 3-71 7, 2002.
Jin, Y., Atkinson, S. J., Marrs, J. A. and Gallagher, P. J. Myosin ii light chain phosphorylation regulates membrane localization and apoptotic signaling of tumor necrosis factor receptor-I. J Biol Chem 276: 30342-30349.200 1.
Kamm, K. E. and Stull, J. T. Dedicated myosin light chain kinases with diverse cellular functions. J. Biol. Chem., 276: 4527-4530, 2001.
Klemke, R. L., Cai, S., Giannini, A. L., Gallagher, P. J., de Lanerolle, P. and Cheresh, D. A. Regulation of cell motility by mitogen-activated protein kinase. J. Cell Biol., 137: 481-492, 1997.
Kimura, K., et al. Science, 273:245, 1996.
Knecht, D. A. and W. F. Loomis. Antisense RNA inactivation of myosin heavy chain gene expression in Dictyostelium discoideum. Science, 236: 1081-1086, 1987.
Kosako, H., Yoshida, T., Matsumura, F., Ishizaki, T., Narumiya, S. and Inagaki, M. Rho-kinase/ROCK is involved in cytokinesis through the phosphorylation of myosin light chain and not ezrin/radixin/moesin proteins at the cleavage furrow. Oncogene, 19: 6059-6064, 2000.
Kothakota, S., Azuma, T., Reinhard, C., Klippel, A., Tang, J., Chu, K., McGarry, T. J., Kirschner, M. W., Koths, K., Kwiatkowski, D. J. and Williams, L. T. Caspase 3-generated fragment of gelsolin: Effector of morphological changes in apoptosis. Science, 278: 294-298, 1997.
Kumar, R. and Vadlamudi, R. K. Emerging functions of p21-activated kinases in human cancer cells. J. Cell Physiol. 193: 133-144, 2002.
Kundra, V., Escobedo, J. A., Kazlauskas, A., Kim, H. K., Rhee, S. G., Williams, L. T. and Zetter, B. R. Regulation of chemotaxis by the platelet-derived growth factor receptor-beta. Nature, 367: 474-476, 1994.
Lee, J. C., Kim, D. C., Gee, M. S., Saunders, H. M., Sehgal, C. M., Feldman, M. D., Ross, S. R. and Lee, W. M. Interleukin-12 inhibits angiogenesis and growth of transplanted but not in situ mouse mammary tumor virus-induced mammary carcinomas. Cancer Res. 62: 747-755, 2002.
Mabuchi, I. and M. Okuno. The effect of myosin antibody on the division of starfish blastomeres. J. Cell Biol., 74: 251-263, 1977.
Matsumoto H, and Sasaki Y, Biochem, Biophys. Res. Commun. 1 58(1): 105-09 (1 989)
Matsumura, F., Totsukawa, G., Yamakita, Y. and Yamashiro, S. Role of myosin light chain phosphorylation in the regulation of cytokinesis. Cell Structure and Function, 26: 639-644, 2001.
Mills, J. C., Stone, N. L., Erhardt, J. and Pittman, R. N. Apoptotic membrane blebbing is regulated by myosin light chain phosphorylation. J. Cell Biol., 140: 627-636, 1998.
Moore et al., Cytotechnology 1 7: 1-11, 1995
Nakanishi et al., 1990; Nakanishi, S., Yamada, K., Iwahashi, K., Kuroda, K., and Kase, H. KT5926, a potent and selective inhibitor of myosin light chain kinase. Mol Pharmacol. 37: 482-488, 1990.
Nakano, T. and Hartshorne, D. J. Regulation of the Contractile Cycle in Smooth Muscle, Springer-Verlag, Tokyo, 1995.
Nam, N. H. and Parang, K. Current targets for anticancer drug discovery. Curr. Drug Targets, 4: 159-1 79, 2003.
Obara, K., Takai, A., Ruegg, J. C., and de Lanerolle, P. (1989), Okadaic acid, a phosphatase inhibitor, produces a Ca2+ and calmodulinindependent contraction of lamb tracheal smooth muscle. Pfluger's Archiv. 41 4: 134-1 38
O'Reilly, M. S., Boehm, T., Shing, Y., Fukai, N., Vasios, G., Lane, W. S., Flynn, E., Birkhead, J. R., Olsen, B. R. and Folkman, J. Endostatin: an endogenous inhibitor of angiogenesis and tumor growth. Cell, 88: 277-285, 1997.
Qu., J., Cammarano, M. S., Shi, Q, Ha, K. C., de Lanerolle, P. and Minden, A. Activated PAK 4 regulates cell adhesion and anchorage independent growth. Mol. Cell. Biol., 21: 3523-33, 2001.
Paul, J. Cell ad Tissue Culture, 5th Edition, Churchill Livingstone, Edinburgh, London and New York
Pestic-Dragovich, L., Stojiljkovic, L., Philimonenko, A., Nowak, G., Ke, Y., Settlage, R. E., Shabanowitz, J., Hunt, D. F., Hozak, P. and de Lanerolle, P. A myosin I isoform in the nucleus. Science, 290: 337-341, 2000.
Petrache, I., Birukov, K., Zaiman, A. L., Crow, M. T., Deng, H., Wadgaonkar, R., Romer, L. H. and Garcia, J. G. Caspase-dependent cleavage of myosin light chain kinase (MLCK) is involved in TNF-alpha-mediated bovine pulmonary endothelial cell apoptosis. FASEB J. 17: 407-1 6, 2003.
Puthalakath, H., Villunger, A., O'Reilly, L. A., Beaumont, J. G., Coultas, L., Cheney, R. E., Huang, D. C. and Strasser, A. Bmf: a proapoptotic BH3-only protein regulated by interaction with the myosin V actin motor complex, activated by anoikis. Science, 293: 1829-1 832, 2001.
Rudel, T. and Bokoch, G. M. Membrane and morphological changes in apoptotic cells regulated by caspase-mediated activation of PAK2. Science, 276: 1571-1574, 1997.
Sanders, L., Matsumura, F., Bokoch, G. M. and de Lanerolle, P. Inhibition of myosin light chain kinase by p21-activated kinase (PAK) during cell spreading. Science, 283: 2083-2085, 1999.
Satterwhite, L. L., Lohka, M. J., Wilson, K. L., Scherson, T. Y. Cisek, L. J., Corden, J. L. and Pollard, T. D. Phosphorylation of myosin-II regulatory light chain by cyclin-p34cdc2: A mechanism for the timing of cytokinesis. J. Cell Biol., 118: 595-602, 1992.
Seasholtz, T. M., et al. Circ. Res., 49:488, 2001.
Sebbagh, M., Renvoize, C., Hamelin, J., Riche, N., Bertoglio, J. and Breard, J. Caspase-3-mediated cleavage of ROCK I induces MLC phosphorylation and apoptotic membrane blebbing. Nat. Cell Biol., 3: 346-352, 2001.
Sehested, M. and Jensen, P. B. Mapping of DNA topoisomerase II poisons and catalytic inhibitors to four distinc ateps in the topoisomerase II catalytic cycle. Biochem Pharmacol. 51: 879-886, 1996
Sells, M. A. and Chernoff, J. Emerging from the Pak: the p21-activated protein kinase family. Trends in Cell Biology, 7: 162-167, 1997.
Skehan, P., Storeng, R., Scudiero, D., Monks, A., McMahon, J., Vistica, D., Warren, J. T., Bokesch, H., Kenney, S., and Boyd, M. R., “New Colorimetric Assay For Anticancer-Drug Screening,” J. Natl. Cancer Inst. 82: 11 07-1 112, 1990
Somlyo, A. P. and Somlyo, A. V. Signal transduction and regulation in smooth muscle. Nature, 372: 231-236, 1994.
Surks, H. K., et al. Science, 286: 1583, 1999.
Tamaoki T., et al., Biochem, Biophys. Res. Commun. 135(2):397-402 (1986)
The Molecular Basis of Cancer, Mendelsohn and Israel, eds., Chapter 1, entitled “Cell cycle regulation, oncogens, and antineoplastic drugs” by Murakami et al., (W B Saunders; Philadelphia, 1995), especially p. 13.
Totsukawa, G., Yamakita, Y., Yamashiro, S., Hartshorne, D. J., Sasaki, Y. and Matsumura, F. Distinct roles of ROCK (Rho-kinase) and MLCK in spatial regulation of MLC phosphorylation for assembly of stress fibers and focal adhesions in 3T3 fibroblasts. J. Cell Biol., 150: 797-806, 2000.
Trybus, K. M. Myosin regulation and assembly. In: Biochemistry of Muscle Contraction ed. by M. Barany (Academic Press, San Diego, Calif.), pp. 37-46, 1996.
Uehata, M., et al. Nature, 389:990, 1997.
U.S. Dept. of Health and Human Services, National Center for Health Statistics, Health United States 1996-97 and Injury Chartbook 1 17 (1997)
Vojtek, A. B., and Cooper, J. A. Rho family members: activators of MAP kinase cascades. Cell, 82:527-529, 1995.
Volberg T., et al., “Effect of protein kinase inhibitor H-7 on the contractility, integrity, and membrane anchorage of the microfilament system,” Cell Motil. Cytoskel. 29:321-338 (1994)
Wilson, A. K., Takai, A., Ruegg, J. C. and de Lanerolle, P. Okadaic acid, a phosphatase inhibitor, decreases macrophage motility. Am. J. Physiol., 260: L105-L112, 1991.
Wilson, A. K., Gorgas, G., Claypool, W. D. and de Lanerolle, P. An increase or a decrease in myosin II phosphorylation inhibits macrophage motility. J. Cell Biol., 1 14: 277-283, 1991.
Wong, Chemistry of protein Conjugation and Cross-linking, 1991, CRC Press, Boca Raton.
Wysolmerski, R. B. and Lagunoff, D. Involvement of myosin light chain kinase in endothelial cell retraction. Proc. Natl. Acad. Sci., USA., 87: 16-20, 1990.
Wysolmerski, R. B. and Lagunoff, D., 1991
Yamakita, Y., Yamashiro, S. and Matsumura, F. In vivo phosphorylation of regulatory light chain of myosin II during mitosis of cultured cells. J. Cell Biol, 124: 129-137. 1994.
Zolotarevsky, Y., Hecht, G., Koutsouris, A., Gonzalez, D. E., Quan, C., Tom, J., Mrsny, R. J. and Turner, J. R. A membrane-permanent peptide that inhibits MLC kinase restores barrier function in vitro models of intestinal disease. Gastroenterology, 123: 163-1 72, 2002.

We claim:

1. A method of treating a patient having neoplasia comprising administering to the patient in need thereof a pharmaceutical composition comprising at least one myosin light chain kinase (MLCK) inhibitor in a therapeutically effective amount sufficient to cause a reduction in MLC-P sufficient to induce apoptosis in neoplastic cells in the patient.

2. The method of claim 1 wherein said neoplasia is breast cancer, prostate cancer or melanoma.

3. The method of claim 1 wherein the myosin light chain kinase inhibitor is ML-9, ML-7, staurosporine, KT-5926, Calphostin C, H-7, H-8, H-89, HA-1 00, HA-1 077, K-252a, K-252b, piceatannol, peptide 18, Sm-I peptide, or peptide 342-352, or a pharmaceutically effective salt, formulation or conjugate thereof, and wherein the neoplasia is breast cancer, prostate cancer, lung carcinoma, renal cell carcinoma, glioma, melanoma, chemotherapy-resistant tumors or metastatic tumors.

4. The method of claim 3 wherein the myosin light chain kinase inhibitor is ML-7, ML-9, or KT-5926.

5. The method of claim 4 wherein the myosin light chain kinase inhibitor is ML-7.

6. The method of claim 1, wherein an adjuvant is further administered to the patient.

7. The method of claim 6, wherein the adjuvant is alum, incomplete Freund's adjuvant, a bacterial capsular polysaccharide, dextran, IL-12, GM-CSF, CD40 ligand, IFN-y, IL-1, IL-2, IL-3, IL-4, IL-10, IL-13, IL-18, a cytokine, or fragments thereof.

8. A method of inducing apoptosis in neoplastic cells in a subject comprising:
administering to the subject a pharmaceutical composition comprising at least one myosin light chain kinase inhibitor in a dose effective to induce apoptosis in the neoplastic cells.

9. The method of claim 8, wherein the MLCK inhibitor is ML-9, ML-7, staurosporine, KT-5926, Calphostin C, H-7, H-8, H-89, HA-I 00, HA-1 077, K-252a, K-252b, Piceatannol, Peptide 18, Sm-I peptide, or Peptide 342-352, or a pharmaceutically effective salt, formulation or conjugate thereof.

10. The method of claim 10, wherein the myosin light chain kinase inhibitor is ML-7, ML-9, or KT-5926.

11. The method of claim 10, wherein the inhibitor is ML-7

12. The method of claim 8, wherein the at least one myosin light chain kinase inhibitor is administered by injection.

13. The method of claim 8, wherein the neoplastic cells are breast cancer cells, prostate cancer cells or melanoma cells.

14. A method of assessing induction of apoptosis in tumor cells in a subject comprising:
a) administering to the subject a pharmaceutical composition comprising an effective amount of at least one myosin light chain kinase inhibitor in a dose effective to induce apoptosis in the neoplastic cells; and
b) evaluating said tumor cells for apoptosis.

15. The method of claim 14, further comprising determining the amount of MLC-P in said tumor cells.

16. The method of claim 14, wherein the inhibitor is ML-9, ML-7, staurosporine, KT-5926, Calphostin C, H-7, H-8, H-89, HA-100, HA-1077, K-252a, K-252b, piceatannol, peptide 18, Sm-I peptide, or peptide 342-352, or a pharmaceutically effective salt, formulation or conjugate thereof.

17. The method of claim 14, wherein the neoplastic cells are breast cancer cells, prostate cancer cells or melanoma cells.

18. A method of inhibiting tumor growth in a subject comprising:
administering to the subject at least one myosin light chain kinase (MLCK) inhibitor in an amount sufficient to cause a reduction in MLC-P sufficient to induce apoptosis in the tumor cells, thereby inhibiting the tumor growth.

19. The method of claim 18 wherein the myosin light chain kinase inhibitor is ML-7, ML-9, or KT-5926.

20. The method of claim 18 wherein the myosin light chain kinase inhibitor is ML-7.

21. The method of claim 18, wherein an adjuvant is further administered to the subject

22. The method of claim 18, wherein the at least one myosin light chain kinase inhibitor is administered by injection.

23. The method of claim 18, wherein the at least one myosin light chain kinase inhibitor is administered orally.

24. A method of treating a patient having neoplasia comprising:
a) administering to the patient in need thereof a pharmaceutical composition comprising at least one cytotoxic drug; and
b) administering to the patient in need thereof a pharmaceutical composition comprising a pharmaceutical composition comprising a compound in a therapeutically effective amount in combination with the cytotoxic drug of part (a) that is sufficient to cause a reduction in MLC-P sufficient to induce apoptosis in neoplastic cells in the patient.

25. The method of claim 24, wherein the compound is an MLCK inhibitor.

26. The method of claim 24, wherein the cytotoxic drug is vincristine, vinblastine, taxol, taxotere, doxorubicin, docetaxel, epirubicin, daunorubicin, etoposide, bleomycin, tamoxifen, prednisone, dacarbazine, mechlorethamine, cisplatin, methotrexate, 5-fluorouracil, or ara-C.

27. The method of claim 24, wherein the cytotoxic drug is etoposide, docetaxel or doxorubicin.

28. A method of treating a patient having neoplasia comprising administering to the patient in need thereof a pharmaceutical composition comprising at least one cytotoxic drug; and administering to the patient in need thereof a pharmaceutical composition comprising at least one myosin light chain kinase inhibitor, in the amounts which, together, are effective to cause a reduction in MLC-P sufficient to induce apoptosis in neoplastic cells in the patient.

29. The method of claim 28, wherein the cytotoxic drug is vincristine, vinblastine, taxol, taxotere, doxorubicin, docetaxel, epirubicin, daunorubicin, etoposide, bleomycin, tamoxifen, prednisone, dacarbazine, mechlorethamine, cisplatin, methotrexate, 5-fluorouracil, or ara-C.

30. The method of claim 29, wherein the cytotoxic drug is etoposide, docetaxel, or doxorubicin.

31. The method of claim 29, wherein the at least one myosin light chain inhibitor is ML-9, ML-7, staurosporine, KT-5926, Calphostin C, H-7, H-8, H-89, HA-100, HA-1077, K-252a, K-252b, piceatannol, peptide 18, Sm-I peptide, or peptide 342-352, or a pharmaceutically effective salt, formulation or conjugate thereof.

35. The method of claim 29, wherein the at least one myosin light chain inhibitor is ML-7.

36. The method of claim 29, wherein the at least one myosin light chain inhibitor is KT-5926.

37. The method of claim 29, wherein the at least one cytotoxic drug and the at least one myosin light chain inhibitor are administered simultaneously.

38. The method of claim 29, wherein the at least one cytotoxic drug is administered before the at least one myosin light chain inhibitor is administered.

39. The method of claim 29, wherein the at least one cytotoxic drug is administered after the at least one myosin light chain inhibitor is administered.

40. A method of inhibiting growth or proliferation of, or inducing reduction in the number of tumor cells in a subject, comprising
administering to the subject at least one cytotoxic agent and at least one MLCK inhibitor, in an amount which, together, is effective to cause a reduction in MLC-P and inhibit growth or proliferation of the tumor cells.

41. A method of inhibiting unwanted growth or proliferation of, or reducing the number of tumor cells in a human subject, comprising administering to the human subject at least one cytotoxic agent and at least one MLCK inhibitor, in an amount, which together, is effective to cause a reduction in MLC-P and reduce or inhibit the growth or proliferation of the established tumor, induce cell death of the established tumors, or to reduce the size of the established tumors.

42. A method of inhibiting unwanted proliferation of, or reducing the size of, an established tumor in a subject, comprising administering to the subject at least one cytotoxic agent and at least one MLCK inhibitor, in an amount, which together, is effective to cause a reduction in MLC-P and reduce or inhibit the growth or proliferation of the established tumor, induce cell death of the established tumors, or to reduce the size of the established tumors, wherein the established tumor is breast cancer, prostate cancer, lung carcinoma, renal cell carcinoma, glioma, melanoma, chemotherapy-resistant tumors or metastatic tumors.

43. A method for identifying a compound for treating neoplasia, comprising:
a) determining an MLC-P level in a first mammalian cell after exposing the cell to the compound;
b) determining whether the compound induces apoptosis of a second mammalian cell; and
c) identifying the compound for treating neoplasia when the compound reduces MLC-P levels in the first mammalian cell and induces apoptosis in the second mammalian cell.

44. The method of claim 43, wherein the first mammalian cell is a cell cultured in vitro.

45. The method of claim 43, wherein the second mammalian cell is a neoplastic cell.

46. The method of claim 43, wherein the compound is a myosin light chain kinase inhibitor.

47. A method of using a compounds identified by the method of claim 43 for treatment of a patient having neoplasia comprising:
administering to the patient in need thereof a pharmaceutical composition comprising at least one said compound in a therapeutically effective amount sufficient to cause a reduction in MLC-P sufficient to induce apoptosis in neoplastic cells in the patient.

48. A method of treating a patient having neoplasia comprising:
administering to the patient in need thereof a pharmaceutical composition comprising at least one compound, identified by the method of claim 43, in a therapeutically effective amount sufficient to cause a reduction in MLC-P sufficient to induce apoptosis in neoplastic cells in the patient.

49. A method of inhibiting a tumor growth in a subject comprising:
administering to the subject at least one compound, identified by the method of claim 43, in an amount sufficient to cause a reduction in MLC-P sufficient to induce apoptosis in tumor cells in the patient, and thereby inhibiting the tumor growth.

50. A method of treating a patient having neoplasia comprising:
administering to the patient in need thereof a pharmaceutical composition comprising at least one cytotoxic drug; and administering to the patient in need thereof a pharmaceutical composition comprising at least one compound, identified by the method of claim 43,
wherein the compounds are administered in amounts sufficient which, together, are effective to cause a reduction in MLC-P sufficient to induce apoptosis in neoplastic cells in the patient.

51. A method for identifying compounds for treating neoplasia n a patient, comprising:
a) determining whether the compound reduces MLC-P levels in the neoplastic cell;
b) determining whether the compound induces apoptosis of a neoplastic cell; and
c) identifying those compounds wherein both MLC-P levels are inhibited and apoptosis is induced in the neoplastic cell.


SEQUENCE LISTING (4 sequences)

SEQ ID NO: 1 - protein, 22 aa, Artificial
  SM-1; MLCK inhibitor peptide 480-501
AKKLSKDRMKKYMARRKWQKTG

SEQ ID NO: 2 - protein, 9 aa, Artificial
  MLCK inhibitor; peptide 18
RKKYKYRRK

SEQ ID NO: 3 - protein, 11 aa, Artificial
  MLCK inhibitor; peptide 342-352
KRRWKKNFIAV

SEQ ID NO: 4 - protein, 4 aa, Artificial
  fluorogenic peptide
DEVD